(19) United States
(12) Patent Application Publication
(10) Pub. No.: US 2019/0359944 A1
(43) Pub. Date: Nov. 28, 2019

(54) CELL PRODUCT OF MAMMALIAN INSULIN-PRODUCING CELLS AND METHODS FOR USING THE SAME

(71) Applicant: PIROGOV RUSSIAN NATIONAL RESEARCH MEDICAL UNIVERSITY (RNRMU), Moscow (RU)

(72) Inventors: Olga Sergeevna PETRAKOVA, Balashikha (RU); Mikhail Aleksandrovich BORISOV, Moscow (RU); Ekaterina Andreevna VOROTELYAK, Moscow (RU); Inessa Givievna GVAZAVA, Moscow (RU); Olga Sergeevna ROGOVAYA, Reutov (RU); Andrey Valentinovich VASILIEV, Moscow (RU); Ekaterina Andreevna BOGDANOVA, Moscow (RU)

(73) Assignee: PIROGOV RUSSIAN NATIONAL RESEARCH MEDICAL UNIVERSITY (RNRMU), Moscow (RU)

(21) Appl. No.: 16/484,293

(22) PCT Filed: Feb. 8, 2018
(86) PCT No.: PCT/RU2018/000078

§ 371 Date: Aug. 7, 2019

(30) Foreign Application Priority Data
Feb. 9, 2017 (RU) 2017104225

(51) Int. Cl. C12N 5/071; A61K 35/39

(52) U.S. Cl. CPC C12N 5/0678; A61K 35/39; C12N 2500/25; C12N 2500/32; C12N 2513/00; C12N 2500/38; C12N 2501/12; C12N 2500/05; C12N 2501/999; C12N 2501/11

(57) ABSTRACT

The technical result of the invention is to simplify the technology of obtaining insulin-producing cells, obtaining at least 70% of functionally active insulin-producing cells in cell culture, that underwent differentiation. The method comprises obtaining epithelial progenitor cells and their subsequent differentiation into pancreatic cells, capable to glucose-sensitive insulin secretion in which pancreatic differentiation is performed in two stages:
(a) at the first stage cells are differentiated within 4-15 days in a culture medium containing at least serum of a mammal, glutamine, epidermal growth factor, transferrin, sodium selenite, retinoic acid, isoproterenol;
(b) at the second stage, cells are differentiated within 4-15 days in a culture medium containing at least serum of a mammal, glutamine, epidermal growth factor, retinoic acid, nicotinamide, hepatocyte growth factor, dexamethasone;
moreover, the cultivation in both stages is carried out in gas atmosphere of 5% CO2 at 37° C. Group of inventions includes cell product of insulin-producing cells of a mammal, and a method of differentiation of pancreatic epithelial progenitor cells of mammals, including humans, as well as a method for replacement therapy of diabetes mellitus using cell product.

### FIELD OF INVENTION

The group of inventions is related to the regenerative medicine and cell technologies.

### BACKGROUND OF THE INVENTION

Diabetes Mellitus is a disease of the endocrine system characterized by glucose malabsorption and resulted from insulin hormone insufficiency. As a consequence, hyperglycemia is progressing, a stable increase in blood glucose level which leads to metabolic disorder of any kind (carbohydrate, lipid, protein, mineral, salt-water). Insulin in mammalian body is produced by beta-cells of endocrine pancreas gathered in anatomical structures called Langerhans islets and characterised by their capability of glucose-dependent insulin secretion. The main types of diabetes mellitus are type I and II diabetes. Type I diabetes mellitus (or achrestic diabetes) is an autoimmune disease of the endocrine system caused by insulin insufficiency resulted from beta-cells disruption by immune cells affected by autoreactive antibodies response to beta-cell proteins. People of juvenile age develop this type of diabetes most often, and namely, children, teenagers, people before 30. Type II diabetes mellitus (insulinindependent diabetes) is a metabolic disease characterised by chronic hyperglycemia associated with reduction in tissue sensitivity to insulin (insulin resistance). At the initial stages of the disease insulin is secreted in increased amounts. In course of time insulin supersecretion exhausts beta-cells. Type II diabetes makes 85-90% of all diabetes mellitus cases and most often advances at people after 40 [Gavin J. R., Davidson M. B., DeFronzo R. A., Drash A, Gabbe S. G., Genuth S., Harris M. I., Kahn R., Keen H., Knowler W. C., Lebovitz H., Maclaren N. K., Palmer J. P., Raskin P., Rizza R. A., Stern M. P. Report of the Expert Committee on the Diagnosis and Classification of Diabetes Mellitus. Diabetes Care. 2003. 26(1). s5-s20].

When developing treatment methods for diabetes and it consequences, experimental diabetes models of laboratory animals are ofter used. Several models are based on genetic traits of laboratory mice that have a genetically conditioned diabetes. [Antoniou A. N., Elliott J., Rosmarakis E., Dyson P. J. MHC class II Ab diabetogenic residue 57 Asp/non-Asp dimorphism influences T-cell recognition and selection. Immunogenetics. 1998. 47(3) 218-225, Driver J. P., Serreze D. V., Chen Y. G. Mouse models for the study of autoimmune type 1 diabetes: a NOD to similarities and differences to human disease. Semin. Immunopathol. 2011. 33. 67-87].

Another experimental model group of diabetes mellitus is represented by chemically-induced diabetes model involving beta-cells selective disruption by chemical substances. In particular, streptozotocin, an antibiotic inducing specific necrosis of beta-cells, can be applied. [Lenzen S. The mechanisms of alloxan- and streptozotocininduced diabetes. Diabetologia. 2008. 51(2). 216-226].

One of the possible ways to overcome the problems of diabetes mellitus is cell replacement therapy realised through replenishment of function of missing beta-cells on glucose-dependent insulin secretion by means of injection of insulin producing cells into the human body.

Human or animal donor cells transplantation methods to people suffering from diabetes mellitus (heterotransplantation) have been described. In particular, there is a work on donor Langerhans islets transplantation to patients with diabetes mellitus [Shapiro A M, et al., N. Engl. J. Med. 2006. 355(13). 1318-1330].

However, such methods have serious restrictions associated with low availability of human donor cells from pancreas and an open question of biological safety of material obtained from animals, including danger of human infection with diseases and/or prions that can occur in pig cells. In addition, xenogeneic transplantation requires immunological for immune system suppression in response to administered foreign biological material. As an alternative to immunological suppression, a number of methods can be suggested that imply placement of foreign cells into special containers from bio-compatible materials which allow nutritional substances and insulin, but do not allow the substances described, for example, in [Skinner S J M, et al., InTech. 2011. V. 11. 391-408, US20040197374 A1 dated 7 Oct. 2004] into the immune system cells. However, such containers are gradually overgrown with connective tissue with formation of fibrous capsule which in the course of time lowers the effectiveness of insulin supply to the body and nutritional substances to the cells and finally leads to their death.

It was suggested to use several methods to obtain insulin-producing cells from embryonic stem cells or from induced pluripotent cells by means of endocrine pancreatic differentiation in vitro. In the work [WO2002092756 A2 21 Nov. 2002] embryonic stem cells are originally cultured on the feeder layer in serum-free medium containing serum substitute, essential amino acids, mercaptoethanol, basic fibroblast growth factor (bFGF). Then the cells are exposed to suspension cultivation on cultural plates with non-adhesive surface in the above stated medium without bFGF. Formed embryonic bodies are disaggregated and placed into culture flasks covered with fibronectin in serum-free medium which are supplemented by the following factors: “insulin-transferrin-selenite” additive, B27 additive, N2 additive, bFGF, laminin and nicotinamide. In 20-30 days insulin secreting cells in glucose dependent manner are derived and stable cell lines are obtained.

Another method [Jiang W, et al., Cell Res. 2007. 17(4). 333-344] stipulates that embryonic stem cells are cultured in serum-free medium, then they are placed into culture flasks covered with 1% matrigel (B&D Biosciences, USA), culture medium is changed to 50% Iscove modified Dulbecco's medium (IMDM) and 50% mixture F12 (F12 Nutriefnt Mixture) supplemented with “insulin-transferrin-selenite” additive, monothioglycerin, albumin. Activin A is added to the cells in two days, retinoic acid is added in four days. In 4 days, culture medium is replaced to DMEM/F12 1:1 supplemented with “insulin-transferrin-selenite” additive, albumin, basic fibroblast growth factor. Nicotinamide is added to the medium in 3 days. In 5 days, cells are spheroidally cultured for maturation for the next 5 days. As a result, cells expressing C-peptide, insulin, glucagon, glucose transporter type 2 (GLUT2) are obtained. By means of the method described, only 15% of cells undergo effective pancreatic differentiation.

There is a method for differentiating stem cells into insulin-producing cells [US20050054102 A1 10 Mar. 2005] based on usage of a number of differentiation media aimed at activation of one or more genes necessary for beta-cells differentiation selected from the group: PDX1, PAX4, PAX6, NGN3, NKX6.1, NKX6.2, NKX2.2, HB9, BETA2, NEUROD, ISI1, HNF1-alpha, HNF1-beta, HNF3 or combination of the genes. Stem cells are first cultured in a plate with non-adhesive surface to form embryoid bodies, then differentiated in IMDM medium supplemented with fetal calf serum, L-glutamine, essential amino acids, epidermal growth factor, basic fibroblast growth factor, progesterone, follistatin and/or activin. As a result, at least 20% of insulin-producing cells are obtained within 15 days, but the obtained culture is heterogeneous and contains different types of cells. A protocol is described for pancreatic differentiation of embryonic stem cells and pluripotent stem cells which makes it possible to obtain functional insulin-producing cells capable of glucose-dependent insulin secretion [Pagliuca F W, et al., Cell. 2014. 159(2). 428-439]. Differentiation of human pluripotent cells is carried out within 28-33 days according to the multistaged protocol.

Cells obtained as a result of this differentiation are capable of glucose-dependent insulin secretion at a high level comparable to mature beta-cells. After transplantation into the body of immunodeficient mice with experimental diabetes (SCID/AKITA), these cells normalize blood glucose level.

A group of methods based on pancreatic differentiation of pluripotent cells (embryonic stem and induced pluripotent cells) has significant drawbacks limiting their usage: first, most methods are charactered by low differentiation efficiency—about 15-40% of cells capable to synthesize insulin. Secondly, when embryonic stem cells are used, their source is material from human embryos, and its production is associated with ethical problems. Thirdly, the resulting cell cultures can only be used for allogeneic transplantation, that is, the lifetime of such cells is limited, and special efforts are required to maintain the inserted insulin-producing cells in the body. Cells with induced pluripotency can be used in autologous version, however, the obtaining methods are long and expensive. Finally, embryonic stem and induced pluripotent cells show tumorigenic properties and tend to form teratomes when introduced into the body.

A method for controlled induction of pancreatic hormone production in non-pancreatic tissues is known [U.S. Pat. No. 6,774,120 B1 10 Aug. 2004]. The method is based on obtaining ectopic expression of PDX1 gene (pancreatic and duodenal homeobox 1). In this method, administration of polypeptide or mRNA of PDX1 had a temporary effect and did not result in stable synthesis of insulin. At the same time, administration of foreign DNA, has certain limitations for medical use, since it carries the risk of mutations, recipient's cell damage and possibility of oncotransformation. Additional problems associated with the use of vector constructions introduced into the recipient's body are the risk of their elimination with time and methylation of the introduced DNA. All this leads to the fact that expression of the target gene decreases or stops in the course of time.

To overcome the mentioned problems, approaches are suggested which stipulate to derive insulin-producing cells from easily-accessible human cell types, such as mesenchymal or epithelial cells.

There is a work on differentiation of mesenchymal cord blood stem cells into insulin-producing cells [Prabakar K R, et al., Cell Transplant. 2012. 21(6). 1321-1339]. The authors used a differentiation protocol which included 5 stages with a total duration of more than 3 weeks. At the first stage, the cells were incubated for 3 days in RPMI medium, activin A and Wnt3a (wingless-type MMTV integration site family, member 3A) were added. At the second stage, fibroblast growth factor 10 (FGF-10) and CYC (3-Keto-N-aminoethyl-amino-caproyl-dihydrocinnamoyl Cyclopamine) were added to RPMI medium with 2% fetal calf serum and cells were incubated for 4 days. At the third stage, the medium was replaced to DMEM supplemented with retinoic acid, CYC, FGF10 and additive B27, cells were incubated for 4 days. At the fourth stage, DAPT (N—[N-(3,5-difluorophenacetyl)-L-alanyl]-S-phenylglycine t-butyl ester) and exendin 4 (Ex4, exendin-4) were added to DMEM medium with B27 additive, cells were incubated for 3 days. At the fifth stage, cells were incubated for 4 days in CMRL medium supplemented with B27 additive, Ex4, hepatocyte growth factor (HGF) and insulin-like growth factor 1 (IGF1). The resulting cells were capable of glucose-dependent secretion of C-peptide in vitro. After transplantation to immunodeficient NOD/SCID mice, human C-peptide was also detected in the animal serum. However, this protocol is rather laborious and time-consuming, since mesenchymal cells used do not belong to the glandular epithelium to which beta-cells of pancreas belong.

There is a work in which the authors used submandibular salivary gland cells histogenetically close to beta-cells to obtain insulin-producing cells [Sato A, et al., Cloning Stem Cells. 2007. 9(2). 191-205]. Pancreatic cell differentiation was induced by spheroid culturing in William's E medium supplemented with 10% fetal calf serum, 10 mM nicotinamide, and 20 ng/ml epidermal growth factor. As a result, on the 6th day of cultivation, cells acquired the ability to express PDX1, insulin and C-peptide. Cells also acquired the ability to glucose-dependent secretion of the C-peptide. However, the epithelial cells differentiation protocol is not optimal in this case and allows obtaining a relatively low level of C-peptide secretion, about 0.5 ng/mg protein, comparable to its background expression in undifferentiated human salivary gland cells.

The closest analogue of the patent one is the method of obtaining insulin-positive cells from CD49f-positive human salivary gland cells cultured in vitro [U.S. Pat. No. 7,659,121 B2 BIOS RES INST INC. 9 Feb. 2010]. The cells used are the cells of glandular epithelium of human salivary glands and can be differentiated into insulin-positive cells in in vitro culture by spheroid cultivation method in the presence of epidermal growth factor (EGF), basic fibroblast growth factor (bFGF) and leukemia inhibitory factor (LIF). To obtain insulin-positive cells, salivary gland cells are cultured under non-adherent conditions to form spheroids in Williams' E medium supplemented with fetal calf serum and glucagon-like peptide-1 (GLP-1). In 7 days, insulin expression was detected in spheroids. These cells can be used to treat pancreatic pathologies. However, the prototype method has serious drawbacks: this approach does not allow to completely differentiate cells in the right direction, resulting in a mixed culture of glucagon- and insulin-positive cells, which indicates the initial stages of differentiation. This work does not demonstrate production of insulin-producing cells capable of glucose-dependent insulin secretion, there are no quantitative data demonstrating the effectiveness of the method used.

Thus, at the present time the problem of obtaining insulin-producing cells for replacement cellular therapy of diabetes mellitus with the use of cells from an easily accessible source and use of simple and non-long differentiation protocols is not solved. This invention is regarded as a solution to the problem.

### SUMMARY OF THE INVENTION

The present invention is directed to a method for obtaining of an insulin-producing mammalian cell product, comprising obtaining epithelial progenitor cells, and their following pancreatic diffirentiation into cells capable of glucose-dependent insulin secretion, in which the pancreatic differentiation is done in two stages:

(a) at the first stage, cells are differentiated during 4-15 days in a culture medium containing at least blood serum of a mammal, glutamine, epidermal growth factor, transferrin, sodium selenite, retinoic acid, isoproterenol;

(b) at the second stage, cells are differentiated during 4-15 days in a culture medium containing at least blood serum of a mammal, glutamine, epidermal growth factor, retinoic acid, nicotinamide, hepatocyte growth factor, dexamethasone. In preferred embodiments, the differentiation culture medium of the first stage contains blood serum—2-20 volume %, glutamine—1-4 mM, epidermal growth factor—1-300 ng/ml, transferrin—0.1-20 mcg/ml, sodium selenite—0.1-20 ng/ml, retinoic acid—0.1-20 μM, isoproterenol—0.1-10 μM.

In preferred embodiments, the differentiation culture medium of the second stage contains blood serum—2-20 volume %, glutamine—no less than 1-4 mM, epidermal growth factor—1-300 ng/ml, transferrin—no less than 0.1 μg/ml, sodium selenite—0.1-20 ng/ml, retinoic acid—10 nM-20 μM, nicotinamide—1-100 mM, hepatocyte growth factor—1-300 ng/ml, dexamethasone—0.01-5 μM.

In some embodiments, the differentiation culture medium of the first stage contains in addition, but not limited to insulin-like growth factor 1, fibroblast growth factor 10, fibroblast growth factor 4 and/or keratinocyte growth factor.

In some embodiments, culture medium at the second stage in addition contains insulin-like growth factor 1 and/or betacellulin.

Epithelial progenitor cells are isolated from the biopsy of the salivary gland, or small intestine, or stomach, or liver, or pancreas.

Cultivation in both stages is carried out at a temperature of 37° C. in CO2 incubator in the presence of 5% CO2. In some embodiments, cultivation is carried out in the presence of 5% CO2 and 5% 02.

In some embodiments progenitor epithelial cells are additionally cultivated before the pancreatic differentiation to increase their biomass.

Unlike the known methods, the proposed differentiation method makes it possible to produce insulin-producing cells from epithelial progenitor cells which can be easily obtained from an adult. These cells form a well-proliferating culture in vitro, they can be easily grown to obtain a large cell mass. The differentiation procedure is simple and is not time-consuming.

The technical result of the invention consists in simplifying the technology of insulin-producing cells production, obtaining at least 70% of functionally active insulin-producing cells in a differentiated cell culture, and is achieved by selecting the optimal conditions for differentiation. The resulting cells produce insulin in a glucose-dependent manner.

Also cell product insulin-producing cells of a mammal, including humans, is provided, obtained by using the method of the present invention, containing at least 1 million cells in 1 ml of isotonic solution or not less than 10 thousand spheroids in 1 ml of isotonic solution. As an isotonic solution using a sterile solution of physiological saline for injection, phosphate buffered saline, Hanks solution, a solution of Versene etc.

Cell product of the present invention can be used for scientific research and for replacement therapy of diabetes mellitus in mammals, including humans. A method for replacement therapy of diabetes mellitus is also provided, including: transplantation of the cell product, containing 50-200 million cells in the body of the recipient suffering from diabetes. In some embodiments of the present invention, the cell product is administered 2-5 times with an interval of 1-6 months. The cell product can be used both in autologous and allogeneic transplant options. In some cases, the cell product is additionally cultivated in three-dimensional conditions and is administered in the form of spheroids.

The administration of cell product into mammalian body, including, human suffering from diabetes mellitus leads to decrease in blood glucose level, decrease in blood glucose concentration discontinuity, and regeneration of pancreatic Langerhans islets.

### BRIEF DESCRIPTION OF THE DRAWINGS

FIGS. 1 and 2 show a result of immunocytochemical staining of human salivary gland progenitor epithelial cells with antibodies to proinsulin (FIG. 1) and insulin (FIG. 2). Cell nuclei (DAPI dye) and proinsulin in cytoplasm (Alexa Fluor 488) are stained.

FIGS. 3 and 4 show a result of immunocytochemical staining of insulin-secreting cells of the cell product with antibodies to proinsulin (FIG. 3) and insulin (FIG. 4). The cell nuclei (DAPI dye) and proinsulin in cytoplasm (Alexa Fluor 488) are stained.

FIGS. 5 and 6 show a result of immonocytochemical staining of a spheroid cryosection with antibodies to proinsulin (FIG. 5) and insulin (FIG. 6). Cell nuclei (DAPI dye) and proinsulin in cytoplasm (Alexa Fluor 488) are stained.

FIG. 7 shows the result of immunocytochemical staining of pancreas cryosection of the experimental Nude mouse on the third day after intraperitoneal transplantation. Cell nuclei (DAPI dye, grey nuclei), human nuclei cells are stained with antibodies to Human nuclei (Alexa Fluor 488, white nuclei), insulin is stained in cytoplasm (Alexa Fluor 546).

FIG. 8 shows a result of immunocytochemical staining of pancreas cryosection of the Nude mouse on the third day after transplantation of the cell product. Cell nuclei (DAPI dye), human nuclei cells are stained with antibodies to Human nuclei (Alexa Fluor 488, white nuclei), macrophage marker CD68 is stained in cytoplasm (Alexa Fluor 546). Cryosection thickness makes 10 μm.

FIGS. 9, 10 and 11 show photographs of histological sections of the pancreas of a healthy mouse (FIG. 9), mouse with streptozotocin induced diabetes (FIG. 10) and mouse with streptozotocin induced diabetes with a transplantation of the cell product (FIG. 11) on the 40th day after the start of the experiment. Staining with hematoxylin-eosin, light microscopy, thickness makes 5 μm.

### DETAILED DESCRIPTION

As indicated above, the present invention provides a method for pancreatic differentiation of mammalian progenitor epithelial cells, including humans, cell product preparation comprising insulin-producing cells for replacement therapy of diabetes mellitus and methods for replacement therapy of diabetes mellitus by correcting blood glucose level when cell product of the present invention is introduced into the body.

The method for differentiation of mammalian epithelial progenitor cells, including human, into cells capable of glucose-dependent insulin secretion is a new method of pancreatic differentiation in vitro and comprise two stages:

(a) at the first stage, cells are cultured during 4-15 days in a culture medium supplemented with at least fetal calf serum and glutamine in the presence of at least the following additives: epidermal growth factor, transferrin, sodium selenite, retinoic acid, isoproterenol;

(b) at the second stage, cells are cultured during 4-15 days in a culture medium supplemented with at least fetal calf serum and glutamine in the presence of at least the following additives: epidermal growth factor, retinoic acid, nicotinamide, hepatocyte growth factor, dexamethasone.

The cell product is an insulin-producing cells obtained by pancreatic differentiation as described above from human epithelial progenitor cells expressing one or more of the markers from the list: c-Kit, Sca-1, EpCAM, LGR-5. After differentiation, cells acquire the ability for glucose-dependent insulin secretion and change their phenotype, as a result one or more of the markers from the list are expressed: PDX1, NGN3, MAFA, NKX6.1, PAX4, PAX6 and insulin. The cell product of the present invention contains at least 70% of insulin-producing cells.

A method for replacement therapy of diabetes mellitus comprises administering the cell product of the present invention to the patients with diabetes mellitus. Administration of cell product leads to decrease in blood glucose level in case of diabetes mellitus, decrease in blood glucose concentration discontinuity, and regeneration of pancreatic Langerhans islets.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the invention components that are described in the publications which might be used in connection with the presently described invention.

### Definitions

The term “marker” or “biomarker” refers to protein or mRNA that is present in a particular cell type and distinguishes it from another cell type. Thus, cells associated with the present invention, are characterised by a specific set of markers.

The terms “progenitor” or “undifferentiated” or similar in regard to cells designate stem cells that are determined to be differentiated into certain cell types (but not terminally differentiated). Progenitor cells have a high proliferative potential when cultured in vitro and have biomarkers that distinguish them from other types of cells.

The term “epithelial” in regard to cells refers to cells derived from epithelial tissues (epithelium), it is a set of differons of polar differentiated cells closely located as a layer on the basement membrane, on the border with the external or internal medium, and also forming the majority of the body's glands. There are two groups of epithelial tissues: superficial epithelium (surface and lining) and glandular epithelium, which is the main tissue of most glands.

The term “differon” refers to the aggregate of cell forms that consist a particular differentiation line, including several different types of cell populations, for example, (stem cells, dividing cells, simple transit cells), i.e. parent-progeny relationship.

The term “mesenchymal” refers to a cell type of mesodermic origin that expresses at least the following markers: CD29, CD44, CD73, CD90, CD105 and is also capable under certain culture conditions of adipogenic, chondrogenic and osteogenic differentiation in vitro.

The term “passage” or “to passage” refers to procedure for adhesive cell cultures removal from cultural dishes (usually using proteolytic enzymes), cells transfer into a suspension state to carry them to new culture dish, followed by cultivation to form adhesive culture. In terms of the present invention, “zero (“0”) passage” in regard to cell culture means incubation period to the first passage, “1 passage” means incubation period after the 1st passage and up to the second passage, etc.

The term “adhesive culture” refers to cells that are in an attached to the surface state.

The term “bioptate” refers to a biological material obtained by biopsy from a donor's body.

The term “cultivation” means a set of methods and protocols by means of which viability and proliferative properties of cells are maintained in vitro.

Cell cultivation is carried out in culture medium. The culture medium is a nutrient medium usually containing a composition of essential amino acids, salts, vitamins, minerals, microelements, sugars, lipids and nucleotides. The culture medium provides cells with the components necessary to meet the nutritional and growth needs of cells. Media that differ in nutrient composition, pH and osmolarity are used for different cell types, cells and cell cultures of different densities. Numerous culture media have been described in the literature. Many media are commercially available, their identification is carried out by name and in some cases catalogue number of the medium. The culture media can be supplemented with any components necessary to maintain the desired cell or cell culture. For example, growth stimulants or cell growth inhibitors, hormones, mammalian blood serum containing growth factors, albumin, globulins and other components can be added to the medium.

The term “cultivation of epithelial progenitor cells” means the cultivation process to increase the biomass of these cells, which does not change the phenotype of these cells.

The term “pancreatic differentiation” is used to refer to the process of cell culture under certain conditions, as a result of which cells become similar to beta-cells of the pancreas. In particular, they experience an expression of characteristic genetic markers such as PDX1, NGN3, MAFA, NKX6.1, PAX4, PAX6 and others, and cells acquire the ability to synthesize insulin. One of the accepted tests demonstrating insulin production by cells is C-peptide detection which is a product formed during insulin maturation from a precursor molecule. The presence of C-peptide in cells suggests that they produce insulin and experience maturation.

The term “pluripotency” refers to ability of cells to differentiate into derivatives of all three germ layers (endoderm, mesoderm, ectoderm). The term “embryonic stem cells” refers to cells derived from the internal cell mass of blastocyte that form a cell culture that retain pluripotent properties during prolonged culture in vitro. The term “cells with induced pluripotency” means cells possessing pluripotent properties derived from somatic cells through epigenetic reprogramming.

The term “spheroid cultivation” means cell cultivation in vitro under non-adherent conditions that allow cells to coalesce into three-dimensional globules containing 500-10000 cells.

The term “isolated” refers to a molecule or cell that is in a medium other than the medium in which the molecule or cell is in natural conditions.

The term “isotonic solution” means solutions, ensuring the following properties: pH from 7.3 to 7.7; osmolality: 280+/−20 mOsm/kg; buffer capacity: not less than 1.4 ml.

The term “confluent monolayer” means a monolayer in which cells cover more than 97% of the surface of the culture flask.

The term “cell product” means a cell culture obtained by the two-stage pancreatic differentiation in vitro, as described above, from the epithelial progenitor cells of mammals, including humans.

### Progenitor Epithelial Cells for Pancreatic Differentiation

For the purposes of pancreatic differentiation of the present invention mammalian progenitor epithelial cells, including mouse, rat, pig, rabbit, human, and the like are used. Progenitor epithelial cells can be obtained from donor cells from various body parts: salivary glands, intestines, stomach, liver, pancreas.

A bioptate containing epithelial progenitor cells is obtained by biopsy or surgical procedure by methods well known in the art. In preferred embodiments, collection of cells and/or tissues during biopsy is performed in vivo. If further use of cells involves medical purposes, then the donor of tissue material should not carry infectious diseases (HIV, hepatitis B and C, syphilis), and also should not have oncological diseases.

The bioptate immediately after collection is transferred under sterile conditions to a Petri dish containing culture medium or isotonic solution and antibiotic. For example, culture media DMEM/F12 1:1, 199, DMEM, IGLA, Alpha-MEM, Ham, F12, IMDM, RPMI-1640, and etc. can be used for the purposes of the present invention, or isotonic solutions: phosphate-buffered saline, Hanks solution, physiological solution, Versene solution, etc. Formulations of isotonic solutions are well known to researchers in the art. As an antibiotic, gentamicin is used in a final concentration of 1-100 μg/ml (e.g., in a final concentration of 40 μg/ml), or other antibiotics: penicillin in a final concentration of 5-200 U/ml (e.g., in a final concentration of 50 U/ml), streptomycin in a final concentration of 5-200 μg/ml (e.g., in a final concentration of 50 μg/ml) or another antibiotic known in the art.

It is known from the state of art that differences in cell isolation conditions do not have a significant effect on the quality of further cell culture and differentiation.

All further manipulations are carried out under sterile conditions that meet GMP (Good Manufacturing Practice) requirements. The epithelial tissue is mechanically ground with sterile instruments (scalpels, tweezers, etc.) to small pieces, for example, about 0.5 to 10 mm³ in size. Then, the pieces of tissue are washed with isotonic solution one to three times, span down by centrifugation, incubated with 0.1-10 mg/ml collagenase type IV (optimally 2 mg/ml collagenase) in DMEM/F12 1:1 medium with 1-4 mM glutamine during 20-60 minutes at 37° C. Cell suspension is then passed through a nylon filter with a pore diameter of 40-100 μm and cells are span down by centrifugation.

Cell centrifuging methods are well known in the art, for example, centrifugation is used for 5-15 minutes at 100-400 g.

Next, a population of cells enriched with epithelial progenitor cells is selected. Various techniques known in the art can be used for this purpose. For example, cells can be sorted by a marker selected from the group: EpCAM, c-Kit, CD49f, LGR5 using magnetic selection or fluorescent selection. For example, cells can be obtained by magnetic separation. In this regard, cell suspension is incubated with antibodies to the selected marker conjugated to magnetic particles. Antibodies to marker protein derived from the same species of animals to which the cell donor belongs are typically used.

When selecting cells derived from salivary gland, liver and pancreas, EpCAM marker is predominantly used, for cells from intestine LGR5 marker is used, for cells from stomach c-Kit marker is used.

Manipulations are carried out according to the instructions of manufacturer of antibodies. For example, incubation is carried out at a temperature of about 4° C. (on ice) during 10-60 minutes, usually 15-40 minutes, using antibodies at a rate of 0.1-10 μg antibody per 106 cells. Magnetic separation on the columns is carried out according to the manufacturer's instructions. Sorted cells are then span down by centrifugation, washed with phosphate-buffered saline and resuspended in culture growth medium.

The resulting cells can be used for pancreatic differentiation or can pass through a culture procedure to increase cell mass of progenitor epithelial cells.

Any method can be used to cultivate cells that allows increasing cell mass of progenitor epithelial cells, which preserves the phenotype and characteristic set of markers for this cell type, ensures homogeneity of cell culture and its proliferative potential.

For example, for culturing cells from salivary gland, the methods described for rat cells [Okumura K. et al., Hepatology. 2003. 38. 104-113], mouse cells [Hisatomi Y. et al., Hepatology. 2004. 39(3). 667-675], pig cells [Matsumoto S., et al., Cloning and Stem Cells. 2007. 9. 176-190], or human cells [WO 2014092575 dated 19 Jun. 2014 “Means and methods for obtaining salivary gland stem cells and use thereof”, Jang S. I. et al., J. Dent. Res. 2015. 94(2). 304-311, WO2004074465 dated 12 Mar. 2009 Human salivary gland-origin stem cell] can be used. For example, the method described in the application [“Human Salivary Gland Cells Culturing Method”, registration number 2016139283, date of entry 6 Oct. 2016] can be used. In this method, cells are cultured in PCT Epidermal Keratinocyte Medium in culture flasks providing cell adhesion at 37° C. in the presence of 5% CO₂ subject to medium replacement every 2-4 days until cells reach the monolayer. Hereupon, cell passage in dilution 1:3-1:5 is carried out which involves removing cells from the surface of the culture flask with trypsin solution in EDTA and transferring them to new culture flasks, and continuing their culture subject to medium replacement every 2 to 4 days during cultivation and passages when cells reach a monolayer in dilution of not more than 1:2-1:3. In preferred embodiments of the method, cells are also incubated in the presence of 5% O₂. In some embodiments of this method, cells immediately after production are incubated for 6-48 hours in DMEM/F12 medium 1:1 containing glutamine in a final concentration of 1-4 mM and fetal calf serum in a final concentration of 5-20% at 37° C. in the presence of 5% CO₂. After that, the medium is changed to PCT Epidermal Keratinocyte Medium. Additionally, insulin, transferrin, sodium selenite, and epidermal growth factor (EGF) can be added to the culture medium.

To cultivate progenitor epithelial cells derived from the liver, pancreas, intestine, stomach, the following method can be used: cells are washed with phosphate-buffered saline and resuspended in a culture medium, for DMEM/F12 1:1 containing 5-20% fetal calf serum, 1% insulin-transferrin-selenite, 1-4 mM glutamine and 1-300 ng/ml epidermal growth factor (EGF). Cells are placed in culture flasks coated with type I collagen in the amount of 5×103 cells per 1 cm² and incubated at 37° C. and 5% CO₂. The culture medium is changed every 3 days. In some embodiments, when cells reach a confluent monolayer (usually 10-15 days after isolation), cells are passaged for further cultivation and cell mass growth. To do this, the culture medium is removed, cells are washed twice with Versene solution, then they are incubated for 5 minutes with 0.05%-0.25% trypsin solution in EDTA in the amount of 1 ml per 25 cm² of the culture flask area. Cells are then washed from trypsin with phosphate-buffered saline, span down by centrifugation for 5-10 minutes at 200 g, diluted in growth medium at a ratio of 1:3, and placed in new culture flasks coated with type I collagen.

The resulting cell culture should contain epithelial progenitor cells for further pancreatic differentiation. For the purposes of the present invention, progenitor epithelial cells can be detected in culture using immunostaining or PCR methods against one or more markers selected from the group: EpCAM, AFP, CD49f, CK18, CK19, LGR5, c-Met. A homogeneous culture containing epithelial progenitor cells, that is, a culture that contains at least 70% of progenitor epithelial cells, more often at least 75% of progenitor epithelial cells, typically 80% or more of progenitor epithelial cells, for 85%, 90%, 95%, 96%, 97%, 98: 99% or more of progenitor epithelial cells, is preferable for medical use.

Progenitor epithelial cells should be capable of proliferation. The level of cell proliferation can, for example, be assessed visually by light microscopy in terms of the number of metaphases per field of microscope view and cell phenotype. In particular, an actively proliferating culture contains at least 3-5% of cells in mitosis state. In case of spontaneous differentiation of culture, the number of cells in mitosis state drops below 3-5%. In addition, undifferentiated actively proliferating epithelial cells have small dimensions (10-30 μm), high nuclear-cytoplasmic ratio, polygonal shape, few processes, and form epithelial layer as cobblestone appearance. In spontaneous differentiation, cells acquire large dimensions (more than 50 μm), often lose contact with each other, acquire a variety of processes, have low nuclear-cytoplasmic ratio and granular cytoplasm. Such cells often have several nuclei.

The proliferation rate can also be estimated from the rate at which cells reach the confluent monolayer. The rate at which cells reach the confluent monolayer depends on the initial cell dilution, but in actively proliferating cells it is higher than in differentiated ones. For example, when cells are diluted at 1:3, the rate at which epithelial cells reach the confluent monolayer makes maximum 15 days, usually 7-10 days. In a differentiated culture, cells reach the confluent monolayer very slowly (more than 15 days) or do not reach it at all.

### Pancreatic Differentiation of Epithelial Progenitor Cells

For pancreatic differentiation according to the method of the present invention, isolated epithelial progenitor cells obtained as described above in the section “Progenitor Epithelial Cells for Pancreatic Differentiation” are used. Cells isolated from the mammalian body, including humans, are used. In some embodiments, these cells are previously undergone the culture procedure.

For pancreatic differentiation, cells are placed in a culture medium supplemented with blood serum of mammals and glutamine. Any eukaryotic cell liquid culture medium that provides calcium ion concentration in the range of 0.5 mM to 2.5 mM, typically in the range of 1-2 mM, for 1.81 mM, can be used as the culture medium. Suitable media, in particular, include DMEM, IMDM, William's E medium, RPMI, Alpha-MEM, 199, MEM, BME. For example, DMEM/F12 1:1 medium can be used.

Low-calcium media are not suitable for cultivation, since they contribute to preservation of undifferentiated state of epithelial progenitor cells.

Mammalian blood serum, for example, fetal calf serum, or serum substitute (KSR, KnockOut Serum Replacement), or autologous serum, is added to the medium in a final concentration of 2-20 volume %, typically 5-15%, for 10%. As used here, the term “blood serum of mammals” includes its synthetic substitutes.

Glutamine is added to the medium in a final concentration of no less than 1 mM, typically 1.5-4 mM, for example 2 mM. The increase in the glutamine concentration above 4 mm does not result in noticeable changes in the condition of the cells and the efficiency of pancreatic differentiation.

Cells are cultured in flasks providing epithelial cell adhesion. For example, culture flasks are pre-coated with type I collagen.

At the first stage of differentiation, cells are cultured in the presence of epidermal growth factor, transferrin, sodium selenite, retinoic acid and isoproterenol. Epidermal growth factor is used in a final concentration of 1-300 ng/ml, often 5-100 ng/ml, usually 8-15 ng/ml, for example, 10 ng/ml. Transferrin is used in a final concentration of no less than 0.1 μg/ml, typically 1-20 μg/ml, for example, 5 μg/ml. Selenite sodium is used in a final concentration of 0.1-20 ng/ml, usually 1-10 ng/ml, for example, 5 ng/ml. Retinoic acid is used in a final concentration of 0.1-20 μM, usually 0.5-5 μM, for example, 2 μM. Isoproterenol is used in a final concentration of 0.1-10 μM, usually 0.2-7 μM, more often 0.5-3 μM, for example, 1 μM.

In some embodiments, a commercially available “insulin-transferrin-selenite” additive is used instead of transferrin and sodium selenite in a concentration providing necessary concentrations of transferrin and sodium selenite (for example, 1 volume %). In this case, culture medium also contains insulin.

In some embodiments, insulin-like growth factor 1 (IGF-1) is also added to the medium. Final concentration of insulin-like growth factor 1 does not exceed 300 ng/ml, and is usually 5-20 ng/ml, for example, 10 ng/ml.

In some embodiments, one of the following factors is also added to the medium: fibroblast growth factor 10, fibroblast growth factor 4, keratinocyte growth factor. Final concentration of selected growth factor 1 does not exceed 300 ng/ml, and is usually 5-20 ng/ml, for example, 10 ng/ml.

Cells are cultured for 4-15 days, usually 5-10 days, for example, 6-8 days. The culture medium is changed to fresh every 1-3 days.

At the second stage of differentiation, cells are cultured in the presence of epidermal growth factor, retinoic acid, nicotinamide, hepatocyte growth factor and dexamethasone. Epidermal growth factor is used in a final concentration of 1-300 ng/ml, often 5-100 ng/ml, usually 8-15 ng/ml, for example, 10 ng/ml. Retinoic acid is used in a final concentration of 10 nM-20 μM, usually 20 nM-1 μM, for example, 100 nM. Nicotinamide is used in a final concentration of 1-100 mM, usually 5-50 mM, for example, 10 mM. Hepatocyte growth factor is used in a final concentration of 1-300 ng/ml, often 5-100 ng/ml, usually 10-50 ng/ml, for example, 20 ng/ml. Dexamethasone is used in a final concentration of 0.01-5 μM, usually 0.05-1 μM, for example, 0.1 μM.

In some embodiments, insulin-like growth factor 1 (IGF-1) is also added to the medium. Final concentration of insulin-like growth factor 1 does not exceed 300 ng/ml, and is usually 5-20 ng/ml, for example, 10 ng/ml.

In some embodiments, betacellulin is also added to the medium. Final concentration of betacellulin does not exceed 300 ng/ml, and is usually 5-50 ng/ml, for example, 20 ng/ml.

Cells are cultured for 4-15 days, usually 5-10 days, for example, 6-8 days. The culture medium is changed to fresh every 1-3 days.

In preferred embodiments of the method, both stages of cell differentiation are carried out at 37° C. in the presence of 5% CO2. In some embodiments, both differentiation stages are carried out in the presence of 5% CO2 and 5% O₂.

### Cell Product for Replacement Therapy of Diabetes Mellitus

Prior to differentiation, epithelial progenitor cells express CD49f and KRT18 markers and one or more of progenitor cell markers from the list: c-Kit, Sca-1, EpCAM, LGR-5. After pancreatic differentiation according to the method of the present invention, at least 99% of cell product cells continue to express CD49f and KRT18 markers.

Also, the cell product of the present invention after pancreatic differentiation contains isolated cells which produce insulin and express markers characteristic of beta-cells of the pancreas. In particular, these cells express characteristic genetic markers such as PDX1, NGN3, MAFA, NKX6.1, PAX4, PAX6 and others. One of the accepted tests demonstrating insulin production by cells is insulin or C-peptide detection which is a product formed during insulin maturation from a precursor molecule. The presence of C-peptide in cells suggests that they produce insulin and experience maturation.

Cell product of the present invention contains at least 70% of such insulin-producing cells, more often less than 75% of insulin-producing cells, typically 80% or more of insulin-producing cells, for example, 85%, 90%, 95%, 96%, 97%, 98%, 99% or more of such cells.

Insulin and/or C-peptide are secreted by the cell product in a glucose-dependent manner, i.e., the level of insulin and/or C-peptide secretion by the cell product increases with glucose concentration increase in the culture medium (in buffer or internal medium of the body after cell product transplantation).

Enzyme-linked immunosorbent assay (ELISA) is used to detect secreted insulin and/or C-peptide. For this purpose, cells are incubated in buffer without glucose for 1-2 hours at 37° C. and 5% CO₂. An isotonic solution, for example, Versene solution, phosphate-buffered saline, physiological saline, Krebs buffer without glucose (containing 59 mM NaCl, 2.35 mM KCl, 0.625 mM CaCl₂, 0.6 mM KH₂PO₄, 0, 6 mM MgSO₄*7H₂O, 12.5 mM NaHCO₃, pH=7.4) can be used as a buffer. After incubation, an isotonic solution is changed to warm (37° C.) Krebs buffer without glucose supplemented with different glucose concentrations (0 mM, 2 mM, 5 mM, 15 mM, 20 mM glucose). The samples are incubated for 1 hour at 37° C. and 5% CO₂. The supernatant is then derived and stored at −70° C. until ELISA is performed. ELISA is performed according to the manufacturer's instructions, for example, using Mercodia Ultrasensitive Insulin ELISA, #10-1132-01 and/or Mercodia Ultrasensitive C-peptide ELISA, #10-1141-01.

In some embodiments, the cell product of the invention cultivated in a three-dimensional manner in the absence of glucose in the culture medium produces at least 100 pM of insulin and C-peptide per 1 million cells, usually at least 150 pM of insulin and C-peptide, more often at least 200 pM of insulin and at least 150 pM of C-peptide. The cell product of the invention cultivated in a three-dimensional manner produces at least 280 pM of insulin and at least 180 pM of C-peptide per 1 million cells in the presence of 5 mM glucose in the culture medium, usually at least 300 pM of insulin and at least 200 pM of C-peptide, more often at least 320 pM of insulin and at least 200 pM of C-peptide. The cell product of the invention cultivated in a three-dimensional manner produces at least 400 pM of insulin and C-peptide per 1 million cells in the presence of 15 mM glucose in the culture medium, usually at least 450 pM of insulin and C-peptide, more often at least 500 pM of insulin and C-peptide.

To detect genetic markers expression specific to a particular cell type, immunodetection with antibodies to selected proteins encoded by these genetic markers or detection by polymerase chain reaction (PCR) can be used. Various variants of PCR detection are well known in the art.

### Modes of Administration of the Cell Product of Mammalian Insulin-Producing Cells

Cells (cell product) obtained during pancreatic differentiation by the methods of the present invention can be used to study biochemical and molecular mechanisms of cell proliferation and differentiation, intercellular interactions, oncotransformation, cytokine expression, cytotoxicity analysis of various substances and etc. Since cells obtained in the invention are not modified cells of the body, they can be used to study genetic expression, signaling pathways that occur in natural biological processes. Thus, these cells can be a model to study mechanisms of epithelial differentiation and intercellular contacts.

Since these cells can be obtained in large quantities, they can be used as a basis to test various substances, for example, to study their effects on biological processes, as well as to assess their effect on cell viability and safety analysis of pharmacological agents.

Cells obtained by the methods of the present invention can be used for autologous or allogeneic transplantation for the purpose of replacement cellular therapy of diabetes mellitus. In preferred embodiments, method of replacement cellular therapy of diabetes mellitus involves taking biopsy sample of epithelial progenitor cells from a cell donor, culturing cells to increase progenitor epithelial cells biomass, two-stage pancreatic differentiation of cells of the present invention, and cells transplantation into a recipient suffering from diabetes mellitus.

Cell transplantation is carried out by methods known in the art, for example, cells are administered as a suspension. In this regard, cells are removed from surface of the culture flask after necessary cell mass increase and pancreatic differentiation: the culture medium is removed, the cells are washed twice with Versene solution, then they are incubated for 5 minutes with 0.05%-0.25% trypsin solution in EDTA in the amount of 1 ml per 25 cm² of the culture flask area. The cells are then washed three times with phosphate-buffered saline, precipitate by centrifugation for 5-10 minutes at 200 g, diluted in a sterile isotonic solution, for example, in physiological saline for injection or in phosphate buffered saline in the amount of 0.5-10 million cells/ml. Cell viability is verified: there must be at least 70% of living cells. Cells are transported and stored at +4° C. not more than 24 hours. Cells are administered into the spleen, or into the portal vein, or the greater omentum or injected abdominally via a syringe in the projection of the pancreas in the amount of 50-200 million per patient. Cells can also be administered fractionally (2-5 times) in the amount of 50-200 million at one administration with an interval of 1-6 months. In addition, cells can be administered as spheroids. For this purpose, on the 10th-15th day of differentiation, cells are removed from the surface of culture flasks and placed in non-adhesive conditions in a medium of the second stage of pancreatic differentiation in the amount of 4-6 thousands of cells per 20 μl medium. Cells are incubated under these conditions for 3-7 days before formation of spheroids which are conglomerates of coalesced cells containing 3-6 thousands of cells. Spheroids are then washed three times with phosphate-buffered saline, percipitated by centrifugation for 5-10 minutes at 100-200 g, diluted in a sterile isotonic solution, for example, in physiological saline for injection or in phosphate-buffered saline in the amount of 0.5-10 million cells/ml (about 0.1-5 thousand spheroids per 1 ml of solution). Spheroids are transported and stored at +4° C. not more than 24 hours. Spheroids are administered into the portal vein, or into the spleen, or into the greater omentum in the amount of 50-200 million cells per patient (about 10-50 thousands of spheroids per patient). Spheroids can also be introduced fractionally (2-5 times) in the amount of 50-200 million cells (about 10-50 thousands of spheroids) with an interval of 1-6 months.

In some embodiments, the replacement therapy method of the present invention includes verification of cell number and viability in cell product after pancreatic differentiation. In some embodiments, the replacement therapy method of the present invention includes detection of markers and C-peptide in cell product.

Cells are counted using methods known to specialists skilled in the art. For example, the number of cells can be estimated by flow cytometry using standard antibodies to specific markers on cell surface, on cell counter, in Gorjaev's chamber or cell sorter.

Cell viability verification is carried out by methods known in the art, for example, by staining with trypan blue. In this regard, cells are removed from the surface of culture flasks by trypsin, washed with phosphate-buffered saline and diluted in phosphate-buffered saline in the amount of 0.1-5 million cells/ml. An aliquot of cell suspension of 50-200 μl is taken and diluted with 4% trypan blue solution (BioRad, USA) in a 1:1 ratio. After 5 minutes, the proportion of living cells in the sample is counted using an automatic cell counter (BioRad, USA) or in Gorjaev's chamber (live cells are not stained with trypan blue). Alternatively, staining can be carried out with a dye that does not penetrate an intact cell membrane, for DAPI or ethidium bromide. Calculation of proportion of stained (i.e., in this case, non-viable) cells is performed visually under a fluorescent microscope or a flow cytofluorimeter.

The following examples are offered by way of illustration and not by way of limitation.

### EXPERIMENTAL PROCEDURE

### Example 1

Pancreatic Differentiation of Human Salivary Gland Progenitor Epithelial Cells

Human submandibular salivary gland bioptate was obtained from a male donor of 37 years old during a planned operation on removal of salivary gland part due to sialolithiasis. The volume of glandular tissue of the bioptate was close to 2 cm³. The bioptate was transferred under sterile conditions to Petri dish with DMEM/F12 1:1 medium (Gibco, USA) and gentamicin 40 μg/ml (PanEco, Russia). All further manipulations were carried out under sterile conditions that meet GMP requirements.

The epithelial tissue of salivary gland was mechanically separated from fat and mesenchymal tissues by sterile instruments under binocular and shredded to small pieces (about 1-5 mm³ in size) by scalpel. The tissue pieces were washed twice with phosphate-buffered saline, span down by centrifugation for 5-10 minutes at 0.8-1.5 thousands rpm, incubated for 30-60 minutes at 37° C. in the presence of 2-4 mg/ml collagenase IV type solution (Gibco, USA) in DMEM/F12 1:1 medium (Gibco, USA) with 2 mM glutamine (Invitrogen, USA). Every 10-15 minutes, tubes with salivary gland pieces were vigorously shaken. After incubation, 10 ml of DMEM/F12 1:1 medium (Gibco, USA) was added to the cells, actively pipetted for 3-5 minutes, then passed through nylon filter with pore diameter of 40-100 μm and the cells were span down by centrifugation for 5-10 minutes at 1-1.5 thousands rpm. Then, magnetic separation of cells was carried out under EpCAM marker, for which purpose cell suspension was washed with 10 ml of phosphate-buffered saline, resuspended in 0.5 ml of phosphate-buffered saline, and the number of cells was counted by use of automated cell counter (Bio-Rad, USA). Hereinafter, salivary gland cells were incubated with anti-human EpCAM antibodies conjugated to magnetic particles (Miltenyi Biotec GmbH, Germany) for 15-40 minutes at +4° C. Antibodies were added from at a rate of 0.1-5 μg per 106 cells. After incubation, cells were washed with 10 ml of phosphate-buffered saline and magnetic separation was performed on MiniMACS™ Separator columns (Miltenyi Biotec GmbH, Germany) according to the manufacturer's instructions. Sorted cells were span down by centrifugation for 5-10 minutes at 200 g, washed with 10 ml of phosphate-buffered saline and resuspended in growth medium PCT Epidermal Keratinocyte Medium (1×, liquid), (CELLnTEC, Switzerland), # CnT-07, containing 1× insulin-transferrin-selenite additive (Invitrogen, USA), 10 ng/ml epidermal growth factor (Sigma, USA). Cells were placed in culture flasks coated with type I collagen in the amount of 5×103 cells per 1 cm2 and incubated at 37° C. and 5% CO2. The culture medium was changed daily during the first 5 days, then it was changed every 3 days.

When cells reached confluent monolayer (on the 10th day after isolation), the culture medium was removed, cells were washed twice with Versene solution (PanEco, Russia), then incubated for 5 minutes with 0.05% trypsin solution in EDTA (Gibco, USA) in the amount of 1 ml per 25 cm2 of the culture flask area. The cells are then washed from trypsin with phosphate-buffered saline, precipitated by centrifugation for 5-10 minutes at 200 g.

For performing pancreatic differentiation cells were resuspended in 1:3 ratio in DMEM/F12 1:1 media (Gibco, USA), containing 10% fetal calf serum (HyClone, USA), 1× insulin-transferrin-selenite additive (Invitrogen, USA), 2 mM glutamine (Invitrogen, USA), 10 ng/ml epidermal growth factor (Sigma, USA with addition of 2 μM retinoeic acid (Sigma, USA), 1 μM isoproterenol (Sigma, USA), 10 ng/ml fibroblast growth factor 10 (FGF-10) (Life Technologies, USA) and 10 ng/ml insulin-like growth factor 1 (IGF-1) (R&D, USA), placed in new culture flasks coated with type I collagen and incubated for 7 days. From 7-th to 14-th day cells were cultivated in DMEM/F12 1:1 media (Gibco, USA) with 10% fetal calf serum (HyClone, USA), 2 mM glutamine (Invitrogen, USA), 10 ng/ml epidermal growth factor (Sigma, USA), with the addition of 100 nM retinoic acid (Sigma, USA), 10 mM nicotinamide (Sigma, USA), 10 ng/ml insulin-like growth factor 1 (IGF-1) (R&D, USA), 20 ng/ml hepatocyte growth factor (HGF) (Gibco, USA) and 0.1 μM dexamethasone (Sigma, USA).

At the end of incubation, total RNA was isolated from cells, which was used for cDNA synthesis and real-time quantitative PCR. Total RNA was isolated using RNeasy Mini Kit (250) (Qiagen, Germany) according to the manufacturer's instructions. cDNA synthesis was then performed using reverse transcriptase MMLV RT kit (Eurogen, Russia) with random primers according to the manufacturer's instructions. After that, quantitative real-time PCR was carried out using PCR mixture qPCRmix-HS SYBR (Eurogen, Russia) according to the manufacturer's instructions on CFX96 Touch™ Real-Time PCR Detection System (Bio-Rad, USA) with gene-specific primers, shown in Table 1. Quantitive PCR data was normalized to GAPDH.

TABLE 1
The list of primers used for
real-time quantitative PCR.

| Gene | | |
|---|---|---|
| short | | |
| name | Gene name | Sequences of primers |
| NGN3 | Neurogenin | SEQ ID NO: 1; SEQ ID NO: 2 |
| | 3 | TAAGAGCGAGTTGGCACTGAGC |
| | | CGTACAAGCTGTGGTCCGCTAT |
| PDX1 | Pancreatic | SEQ ID NO: 3; SEQ ID NO: 4 |
| | and | ATAAGAGGACCCGTACAGCTT |
| | duodenal | CTCCGTCAAGTTGAGCATCAC |
| | homeobox 1 | |
| MAFA | MAF bZIP | SEQ ID NO: 5; SEQ ID NO: 6 |
| | tran- | GAGCGGCTAQCCAGCATCAC |
| | scription | CTCTGGAGTTGGCACTTCTCG |
| | factor A | |
| PAX4 | Paired | SEQ ID NO: 7; SEQ ID NO: 8 |
| | box 4 | CTACCGCACAGGTGTCTTGG |
| | | CGTTGGATTTCCCAGGCAA |
| PAX6 | Paired | SEQ ID NO: 9; SEQ ID NO: 10 |
| | box 6 | TGGGCAGGTATTACGAGACTG |
| | | ACTCCCGCTTATACTGGGCTA |
| NKX6.1 | NK6 | SEQ ID NO: 11; SEQ ID NO: 12 |
| | homeobox 1 | ACACGAGACCCACTTTTTCCG |
| | | TGCTGGACTTGTGCTTCTTCAAC |
| GCG | Glucagon | SEQ ID NO: 13; SEQ ID NO: 14 |
| | | CTGAAGGGACCTTTACCAGTGA |
| | | CCTGGCGGCAAGATTATCAAG |
| PPY | Pancreatic | SEQ ID NO: 15; SEQ ID NO: 16 |
| | polypeptide | CCTGCGTGGCTCTGTTACTAC |
| | | CCTGGTCAGCATGTTGATGTATC |
| SST | Somato- | SEQ ID NO: 17; SEQ ID NO: 18 |
| | statin | ACCCAACCAGACGGAGAATGA |
| | | GCCGGGTTTGAGTTAGCAGA |
| AMY2A | Amylase, | SEQ ID NO: 19; SEQ ID NO: 20 |
| | alpha 2A | AATACACAACAAGGACGGACATC |
| | | TCCAAATCCCTTCGGAGCTAAA |
| INS | Prepro- | SEQ ID NO: 21; SEQ ID NO: 22 |
| | insulin | TTTGTGAACCAACACCTGTG |
| | | AGTTGCAGTAGTTCTCCAGC |
| HNF4a | Hepatocyte | SEQ ID NO: 23; SEQ ID NO: 24 |
| | nuclear | ATCTGCGATGCTGGCAATCT |
| | factor | CGAAGGTCAAGCTATGAGGACA |
| | 4 alpha | |
| GLUT2 | Solute | SEQ ID NO: 25; SEQ ID NO: 26 |
| | carrier | GTGTTCCACTGGATGACCGAA |
| | family 2 | AGAATGATGCAGTCATTCCACC |
| | member 2 | |

PCR analysis showed that after pancreatic differentiation of human salivary gland cells, they increase expression of key transcription factors necessary for beta-cells differentiation: NGN3, PDX1, MAFA, PAX4, PAX6, NKX6.1 (Table 2). In addition, after pancreatic differentiation, RNA preproinsulin expression increases (Table 2), whereas amylase expression decreases. Thus, it was concluded that proposed protocol ensures specifically and effectively endocrine differentiation of epithelial cells.

TABLE 2
Result of PCR analisys of gene expression changes after pancreatic
differentiation of human salivary gland cells.

| | Before | |
|---|---|---|
| Gene short name: | differentiation | After differentiation |
| NGN3 | 0.011 | 0.7 |
| PDX1 | 0.02 | 0.47 |
| MAFA | 0.002 | 0.35 |
| PAX4 | 0.004 | 0.043 |
| PAX6 | 0.003 | 0.13 |
| NKX6.1 | 0.0011 | 0.08 |
| GCG | 0.0002 | 0.008 |
| PPY | 0.0003 | 0.011 |
| SST | 0.0002 | 0.008 |
| AMY2A | 0.17 | 0.05 |
| INS | 0.002 | 0.14 |

In order to evaluate the efficiency of pancreatic differentiation epithelial progenitor cells of the salivary gland after 1st passage and cells after pancreatic differentiation were investigated. Cells were stained with antibodies to insulin and proinsulin (a list of the used antibodies are listed in table 3). 48 hours prior to staining, the undifferentiated cells were seeded into I type collagen coated culture flasks and cultivated on PCT Epidermal Keratinocyte Medium (1×, liquid) (CELLnTEC, #CnT-07, Switzerland), containing 1× insulin-transferrin-selenite supplement (Invitrogen, USA) and 10 ng/ml epidermal growth factor (Sigma, USA), while differentiated cells were cultivated on DMEM/F12 1:1 medium (Gibco, USA), containing 10% fetal calf serum (HyClone, USA), 2 mM glutamine (Invitrogen, USA), 10 ng/ml epidermal growth factor (Sigma, USA) with addition of 100 nM retinoic acid (Sigma, USA), 10 mM nicotinamide (Sigma, USA), 10 ng/ml insulin-like growth factor 1 (R&D, USA), 20 ng/ml hepatocyte growth factor (Gibco, USA) and 0.1 μm dexamethasone (Sigma, USA). Then the culture medium was removed, the cells were washed with phosphate-buffered saline and fixed with 4% paraformaldehyde (Sigma, USA) during 10 minutes at room temperature. The cells were washed thrice with phosphate-buffered saline, then blocking of non-specific binding of antibodies in 1% fetal calf serum (Sigma, USA) and 0.1% triton solution (Sigma, USA) was carried out in phosphate-buffered saline at room temperature during 30 minutes. With primary antibodies, the cells were incubated in phosphate-buffered saline for 60 minutes at 37° C. (or at +4° C. overnight) at a dilution recommended by the manufacturer (usually 1: 200-1:500). The cells were washed with phosphate-buffered saline three times for 10 minutes at a time at 37° C., then incubated in phosphate-buffered saline with secondary antibodies (dilution at the ratio of 1:1000) at 37° C. during 40-60 minutes. Then the cells were washed again at 37° C. with phosphate-buffered saline three times for 10 minutes at a time, adding 1 μg/ml DAPI (Sigma, USA). The cells were analyzed under Olympus IX51 (Olympus, Japan) fluorescent microscope.

TABLE 3
The list of antibodies used

| | | |
|---|---|---|
| | | Manufacturing |
| Antibody name: | Antigen: | company and country: |

Primary antibodies:

| | | |
|---|---|---|
| anti-Amylase | Amylase | AbCam, Great Britain |
| anti-c-Kit | Kit tyrosine kinase | Millipore, USA |
| anti-CD68 | Cluster of differentiation | AbCam, Great Britain |
| | 68, macrophage marker | |
| anti-EpCAM | EpCAM | Miltenyi Biotec GmbH, |
| | | Germany |
| anti-Human nuclei | Human core protein | Millipore, USA |
| anti-Insulin | Insulin | AbCam, Great Britain |
| anti-LGR-5 | Receptor 49 binding | AbCam, Great Britain |
| | G-protein | |
| anti-Proinsulin | Proinsulin | AbCam, Great Britain |

Secondary antibodies:

| | |
|---|---|
| Alexa Fluor ® 488 donkey anti-rabbit IgG | Invitrogen, USA |
| (H + L) | |
| Alexa Fluor ® 546 goat anti-rabbit IgG | Invitrogen, USA |
| (H + L) | |

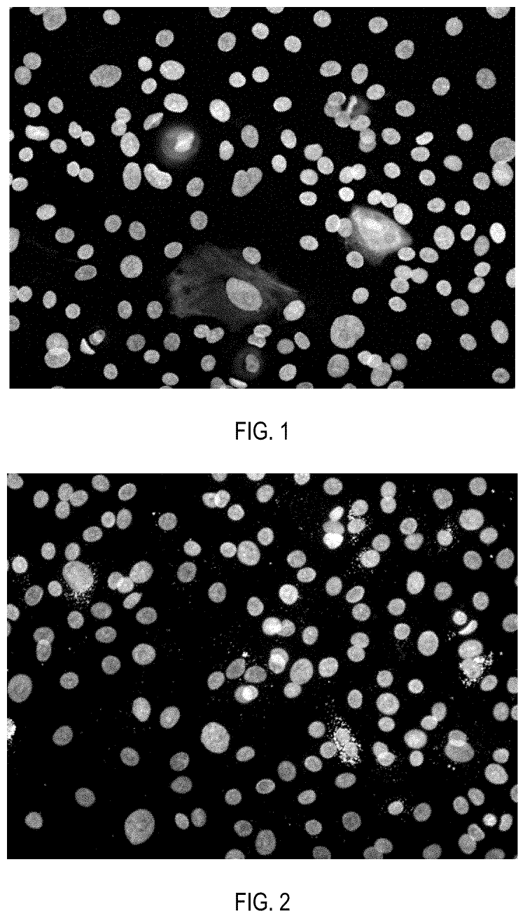

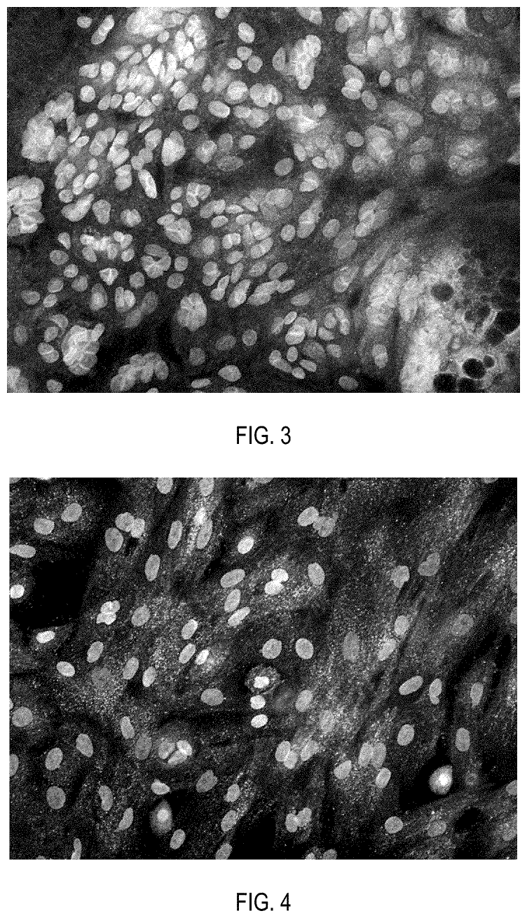

The increase of production of proteins of proinsulin and insulin in the cell product after differentiation compared with the initial culture of progenitor epithelial cells has been shown (FIG. 1-4).

Quantitative assessment of changes in the level of insulin and proinsulin expression was performed by flow cytometry. For this purpose, the culture medium was removed, the cells were washed twice with Versene solution (PanEco, Russia), then incubated in EDTA solution with addition of 0.05% trypsin (Gibco, USA) for 5 minutes. Trypsin solution was added in amount of 1 ml per 25 cm² of a culture flask surface area. Then the cells were washed from trypsin by phosphate-buffered saline, pelleted at 200 g for 5-10 minutes and resuspended thoroughly in phosphate-buffered saline with 2% fetal calf serum (HyClone, USA) in order to obtain monocellular suspension. The cell suspension was divided into aliquots (1×10⁶ of cells per antibody plus isotype controls) and incubated with primary antibodies at the manufacturer's recommended dilution ratio (1:500-1:1000) at room temperature in darkness for 60 minutes. Then the cells were washed with phosphate-buffered saline three times for 10 minutes at a time, and in case when antibodies were conjugated to fluorochrome, they were fixed with 1% paraformaldehyde (Sigma, USA) in darkness for 5 minutes. Afterwards, the cells were washed thrice with phosphate-buffered saline, resuspended in 1 ml of phosphate-buffered saline and analyzed by Cell Lab Quanta™ SC MPL flow cytometer (Beckman Coulter). In case when primary antibodies were not conjugated to fluorochrome, the cells after being washed from the primary antibodies were incubated with the secondary antibodies at room temperature in darkness for 60 minutes. Then the cells were fixed with 1% paraformaldehyde (Sigma, USA) and analyzed as described above. The relevant isotype control was used and at least 10,000 cells were analyzed.

Flow cytometry showed that after the differentiation of human salivary gland cells, the proportion of cells expressing the protein of proinsulin is increased from 32% to 77%, and the protein insulin from 5% to 88%. The changes in gene expression resulting from the application of the developed protocol of pancreatic differentiation are unique, and are not described for other protocols and other cell types.

The influence of various factors on the effectiveness of pancreatic differentiation was investigated. Human salivary gland cells obtained as described earlier were used in the experiments. To conduct pancreatic differentiation concentrations of individual components of the culture media were varied. At the end of differentiation, total RNA was isolated from the cells, cDNA synthesis and efficiency analysis was performed by real-time quantitative PCR. Human salivary gland cells which were not differentiated or differentiated according to protocol described earlier were used as a means of control.

The possibility of using other liquid culture media was tested for pancreatic differentiation. The following media were used: DMEM (Gibco, USA), IMDM (Gibco, USA), MEM (Gibco, USA), William's E medium (Gibco, USA), RPMI 1640 (Gibco, USA), Alpha-MEM (Gibco, USA), PCT Epidermal Keratinocyte Medium # CnT-07 (1×, liquid), (CELLnTEC, Switzerland). As a result of PCR analysis, there was no significant difference between expression of endocrine pancreatic differentiation genetic markers when differentiation was performed in the culture media used, except for Epidermal Keratinocyte Medium # CnT-07, in which elevation of expression level of amylase exocrine marker was observed. It was concluded that DMEM, IMDM, MEM, William's E medium, RPMI 1640 and Alpha-MEM medium could be used for carrying out pancreatic differentiation.

It was shown that when concentration of fetal calf serum is lower than 2 volume % in the differentiating medium, the mitotic activity is not observed in the cells, differentiation efficiency decreases, which results in decrease of expression of beta-cell markers (PDX1, NGN3, MAFA, NKX6.1, PAX4, PAX6, INS). When concentration of fetal calf serum increases above 20 volume % in the differentiation medium, cell death is observed.

When glutamine concentration in the medium decreases below 1 mM, mitotic activity is not observed in cells and there are signs of cell fasting (large bright nucleus, reduction in protein synthesis). The cell differentiation effectiveness is also reduced. When glutamine concentration in the medium increases above 4 mM, there is no significant change in cell behaviour and cell differentiation effectiveness, therefore it is impractical to increase the recommended concentration.

When epidermal growth factor concentration decreases below 1 ng/ml, mitotic activity of cells and pancreatic differentiation effectiveness decreased, which is expressed by decrease in level of expression of beta-cell markers (PDX1, NGN3, MAFA, NKX6.1, PAX4, PAX6, INS). When epidermal growth factor concentration increases above 300 ng/ml, mitotic activity of cells and cell differentiation are also decreased.

When transferrin concentration decreases below 0.1 μg/ml, cell viability decreases, whereas increase in its concentration above 20 μg/ml did not significantly affect the cells.

Reduction in sodium selenite concentration below 0.1 ng/ml leads to decrease in cell proliferative activity, as well as increase in spontaneous differentiation, which is manifested in increase in cell size, appearance of cytoplasm granularity. There are signs of cell culture degradation, cell death increases, and pancreatic differentiation efficiency decreases. Exceeding sodium selenite concentration above 20 ng/ml leads to decrease in pancreatic differentiation effectiveness.

Both lowering retinoic acid concentration below 0.1 μM and increasing its concentration above 20 μM at the first stage of differentiation lead to significant decrease in epithelial cells pancreatic differentiation effectiveness, as evidenced by almost complete absence of increase in pancreatic markers expression (PDX1, NGN3, MAFA, NKX6.1, PAX4, PAX6, INS). At the second stage of pancreatic differentiation, decrease in retinoic acid concentration below 10 nM and its increase above 20 μM also significantly reduce pancreatic differentiation effectiveness.

Reduction in isoproterenol concentration below 0.1 μM at the first stage of differentiation significantly reduces expression of PAX4 required for development of beta-cells, which leads to decrease in pancreatic differentiation effectiveness of epithelial cells. Increase in isoproterenol concentration above 10 μM at the first stage of differentiation had a deterrent effect on differentiation, that resulted in decrease of expression levels of pancreatic markers.

At the second stage of differentiation, decrease in nicotinamide concentration below 1 mM leads to decrease in level of expression of pancreatic markers (PDX1, NGN3, MAFA, NKX6.1, PAX4, PAX6, INS). Increase in nicotinamide concentration above 100 mM decreased cell viability

Reduction of the concentration of hepatocyte growth factor on the second stage of differentiation below 1 ng/ml, as well as lowering the concentration of dexamethasone is below 0.01 μm led to a decrease in the level of gene expression of preproinsulin (INS). Increased concentration of hepatocyte growth factor above 300 ng/ml of dexamethasone or above 5 μm depressed cell proliferation and reduced cell viability.

It was shown as well, that the use of insulin-transferrin-selenite additive at the first stage of differentiation has practically no effect on pancreatic markers expression by epithelial cells in comparison to use of transferrin and sodium selenite.

Addition of insulin-like growth factor 1 to the culture medium on the first and second stages of differentiation increased the viability of epithelial cells. When adding fibroblast growth factor 10, fibroblast growth factor 4 or the growth factor of keratinocytes; at the first stage of differentiation an increase in the viability and mitotic activity of cells was observed. The use of betacellulin on the second stage of differentiation has led to a slight increase in expression of preproinsulin. The conclusion was made about the possibility of adding these components into the culture medium when conducting pancreatic differentiation.

The effect of gas mixture on epithelial cells differentiation was studied: differentiation effectiveness was evaluated in the presence of 5% CO₂, and also in the presence of 5% CO₂ and 5% O₂. It was shown that pancreatic differentiation occurs in both gas mixtures studied, however, in the presence of 5% CO₂ and 5% O₂ cell viability increases: spontaneous cell death decreases. For this reason, in some embodiments, it is possible to use a gas mixture of 5% CO₂ and 5% O₂.

The influence of differentiation stages duration was also studied. It was shown that if the first and second stages of pancreatic differentiation last less than 4 days, cells do not have time to differentiate, which results in the absence of pancreatic markers expression: PDX1, NGN3, MAFA, NKX6.1, PAX6, INS. If the first and second stages of pancreatic differentiation last more than 15 days, cell viability and cell proliferative activity decreases and their spontaneous death increases.

### Example 2

Pancreatic Differentiation of Human Liver, Pancreas, Small Bowel and Stomach Epithelial Progenitor Cells

Human liver, pancreas, small bowel and stomach bioptic samples were extracted in the course of planned surgery to remove organ parts. 0.5-5 cm³ bioptic samples were aseptically transferred into Petri dish containing DMEM/F12 1:1 medium (Gibco, USA) and 40 μg/ml gentamycin (PanEco, Russia). All further manipulations were carried out in sterile conditions compliant with GMP requirements.

Organ biotic samples were washed thrice with phosphate-buffered saline, mechanically divided by sterile tools (scalpel, forceps) into 1-5 mm³ fragments. Then tissue fragments were pelleted during 5-10 minutes at 0.8-1.5 thousand revolutions per minute and incubated with addition of 2-4 mg/ml IV type collagenase (Gibco, USA) in DMEM/F12 1:1 medium (Gibco, USA) containing 1-4 mM of glutamine (Invitrogen, USA) (total—1 ml of solution per 0.5 cm³ of tissue). Tubes with tissue fragments were actively shaken every 10-15 minutes.

After that, 10 ml of DMEM/F12 1:1 medium (Gibco, USA) was added with active pipetting during 3-5 minutes and infiltration through nylon filter with pores 40-100 μm in diameter, then the cells were pelleted during 5-10 minutes at 0.8-1.5 thousand revolutions per minute.

The cells were magnetically separated by EpCAM marker to obtain liver and pancreas progenitor cells. For this purpose, the cell suspension was washed with 10 ml of phosphate-buffered saline, resuspended in 0.5 ml of phosphate-buffered saline, after that the cells were counted by use of automated cell counter (Bio-Rad, USA). Then the cells were incubated with anti-human EpCAM antibodies conjugated with magnetic particles (Miltenyi Biotec GmbH, Germany) at +4° C. during 15-40 minutes. Antibodies were added in amount of 0.1-5 μg per 10⁶ cells. After incubation the cells were washed with 10 ml of phosphate-buffered saline and magnetically separated on columns MiniMACS™ Separator (Miltenyi Biotec GmbH, Germany) according to the manufacturer's instructions. Sorted out cells were pelleted during 5-10 minutes at 200 g.

Fluorescence-activated cell sorting by LGR-5 marker was applied to obtain progenitor bowel cells and by c-Kit marker to obtain progenitor stomach cells. The cell suspension was washed with 10 ml of phosphate-buffered saline, resuspended in 0.5 ml of phosphate-buffered saline, after that the cells were counted by use of automated cell counter (Bio-Rad, USA). Then the cells were incubated with anti-LGR-5 antibodies (or anti-c-Kit respectively) conjugated with Alexa Fluor 488 fluorescent tag at +4° C. during 15-40 minutes. Antibodies were added in amount of 0.1-5 μg per 10⁶ cells. The cells were washed with 10 ml of phosphate-buffered saline and afterwards fluorescence-activated cell sorting was performed using S3e™ Cell Sorter (Bio-Rad, USA).

Liver, pancreas, bowel and stomach sorted cells were washed with 10 ml of phosphate-buffered saline and resuspended in DMEM/F12 1:1 growth medium (Gibco, USA) containing 10% fetal calf serum (HyClone, USA), 1× insulin-transferrin-selenite supplement (Invitrogen, USA), 2 mM glutamine (Invitrogen, USA) and 10 ng/ml epidermal growth factor (Sigma, USA). The cells were seeded into I type collagen coated culture flasks in amount of 5×10³ cells per cm² and incubated at 37° C. and 5% CO₂, providing medium change every 3 days.

After human epithelial cells of liver, pancreas, small bowel and stomach reached monolayer during the first passage they were subjected to pancreatic differentiation by the protocol described in Example 1.

On the 14^{th} day of differentiation, total RNA isolation from cells, complementary DNA synthesis and pancreatic cell differentiation genetic markers analyses were carried out using qRT-PCR method as described in Example 1. The corresponding human epithelial cells which did not undergo differentiation were used as controls in the first passage. Data of the qPCR were normalized by GAPDH.

Human epithelial cell pancreatic differentiation PCR analysis results showed the effectiveness of the protocol used for all studied cell types (Table 6). After endocrine pancreatic differentiation, increase in gene expression of transcription factors necessary for insulin-producing cells (NGN3, PDX1, MAFA, PAX4, NKX6.1) and preproinsulin formation was observed. There was no significant increase in the expression of amylase, somatostatin, glucagon, pancreatic polypeptide, which indicated the specificity of the protocol used for all cultures studied.

TABLE 4
Results of PCR analisys of levels of gene expression
before and after pancreatic differentiation

| Gene | | | | | | | | |
|---|---|---|---|---|---|---|---|---|
| short | Pancreatic cells | | Liver cells | | Stomach cells | | Bowel cells | |
| name: | Before | After | Before | After | Before | After | Before | After |
| NGN3 | 0.01 | 0.7 | 0.0001 | 0.5 | 0.0001 | 0.5 | 0.0001 | 0.7 |
| PDX1 | 0.01 | 0.6 | 0.0001 | 04 | 0.0001 | 0.55 | 0.0001 | 0.3 |
| MAFA | 0.003 | 0.4 | 0.0001 | 0.25 | 0.0001 | 0.2 | 0.0001 | 0.2 |
| PAX4 | 0.003 | 0.2 | 0.003 | 0.04 | 0.05 | 0.3 | 0.006 | 0.4 |
| PAX6 | 0.001 | 0.1 | 0.004 | 0.07 | 0.01 | 0.1 | 0.004 | 0.3 |
| NKX6.1 | 0.007 | 0.4 | 0.0001 | 0.15 | 0.0001 | 0.12 | 0.0001 | 0.09 |
| GCG | 0.0007 | 0.003 | 0.0001 | 0.0002 | 0.0001 | 0.0001 | 0.0001 | 0.0001 |
| PPY | 0.0007 | 0.002 | 0.0001 | 0.0001 | 0.0001 | 0.0001 | 0.0001 | 0.0001 |
| SST | 0.0025 | 0.007 | 0.0001 | 0.004 | 0.0001 | 0.0001 | 0.0001 | 0.0001 |
| AMY2A | 0.002 | 0.007 | 0.0001 | 0.0003 | 0.0001 | 0.0003 | 0.0001 | 0.0001 |
| INS | 0.0001 | 0.315 | 0.0001 | 0.1 | 0.0001 | 0.12 | 0.0001 | 0.11 |

### Example 3

Cultivation of Cell Product in Conditions of 3D Cultivations

Cell product of insulin-secreting cells, produced as described in Example 1, after pancreatic differentiation was washed twice with Versene solution (PanEco, Russia), then incubated in EDTA solution with addition of 0.05% trypsin (Gibco, USA) for 5 minutes. Trypsin solution was added in amount of 1 ml per 25 cm² of a culture flask surface area. Then the cells were washed from trypsin by phosphate-buffered saline, pelleted at 200 g for 5-10 minutes and resuspended in the same medium used for the second stage of pancreatic differentiation. The medium was added in amount of 20 μl of medium for every 5,000 cells. 20 μl of the cell suspension were pipetted on the Petri dish lid, then the dish was closed and the cells were aseptically incubated as hanging drops at 37° C. and 5% CO₂ for the next 5 days. As a result, cells inside the droplets formed aggregates in the form of spheroids of 250-500 μm in size.

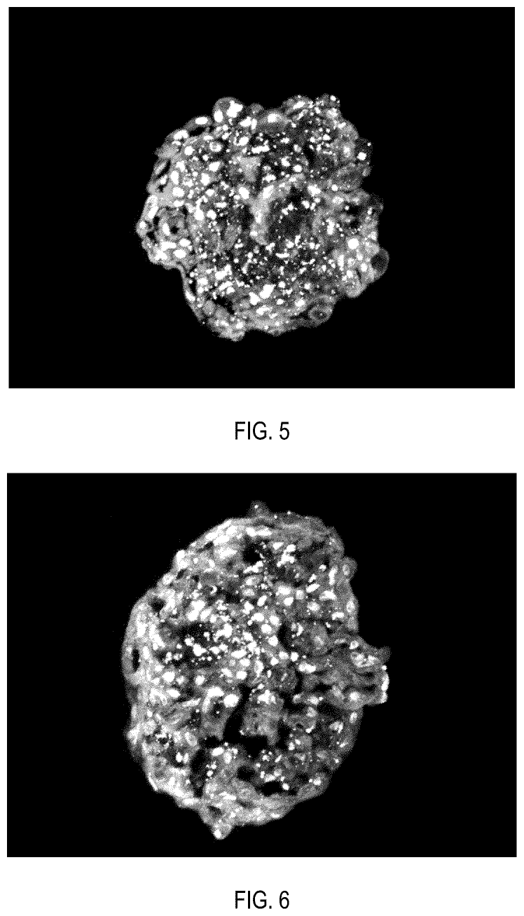

Immunocytochemisty method was used for analysis of insulin and proinsulin expression in spheroids. The spheroids were pipetted into a test tube, pelleted at 200 g for 5 minutes, the supernatant was removed, the residue was frozen in the medium for preparation of OCT cryosections (CrioMount Histolab, Sweden). Cryosections were prepared with the use of Leica CM1900 cryostat (Leica, Switzerland) on Super Frost Plus slides (Menzel, Germany) with a section thickness of 10 μm. Immediately after the preparation, cryosections were fixed with 4% paraformaldehyde (Sigma, USA) at room temperature for 10 minutes. Then they were washed with phosphate-buffered saline and incubated with primary antibodies to insulin and proinsulin at a dilution of 1:500 in 0.1% triton solution (Sigma, USA) and 1% fetal calf serum (Sigma, USA) in phosphate-buffered saline overnight at +4° C. Cryosections were washed with phosphate-buffered saline three times for 10 minutes at a time at 37° C. and incubated with secondary antibodies at a dilution of 1:1000 at 37° C. during 60 minutes. Cryosections were washed with phosphate-buffered saline three times for 10 minutes at a time and the cell nuclei were stained with 1 μg/ml DAPI (Sigma, USA) for 5 minutes. The cryosections were then washed with phosphate-buffered saline and enclosed under a cover glass in 25% glycerin (Sigma, USA) and 25% polyethylene glycol (Sigma, USA) medium in phosphate-buffered saline. The sections were analyzed under Keyence BZ-9000 fluorescent microscope (Keyence, Japan). Immunocytochemical staining of spheroid cryosections of salivary gland cells showed that differentiated epithelial cells actively expressed proteins of proinsulin (FIG. 5) and insulin (FIG. 6) in three-dimensional culture conditions.

Analysis of gene expression required for glucose-dependent insulin secretion by quantitative real-time PCR was carried out as described in Example 1. Undifferentiated epithelial progenitor cells and cells after pancreatic differentiation cultured in two-dimensional conditions were used as controls. It was shown that human progenitor epithelial cells that had not undergone differentiation practically did not express mRNA genes necessary for glucose-dependent secretion of insulin. After differentiation the expression level of these genes increased, moreover, the expression level was higher in cells that had been cultured under three-dimensional conditions (Table 5). Data of the qPCR were normalized by GAPDH.

TABLE 5
Results of the PCR analisys of genes expression levels before and after
pancreatic differentiation and after three dimentional cultivation.

| | | | After three |
|---|---|---|---|
| | Before | After | dimentional |
| Gene short name: | differentiation | differentiation | cultivation |
| GLUT2 | 0.0006 | 0.0005 | 0.018 |
| GLP1R | 0.0008 | 0.008 | 0.097 |
| SYCN | 0.0004 | 0.0009 | 0.048 |
| SNAP25 | 0.0004 | 0.002 | 0.087 |

Investigation of the secretion of insulin and C-peptide in response to different concentrations of glucose was performed by the method of enzyme-linked immunosorbent assay (ELISA) for salivary gland cells that had undergone spheroid cultivation. For this purpose the spheroids were prepared according to the method described above. Spheroids were collected by a pipette from the Petri dish lids, pelleted at 200 g for 5 minutes and washed thrice with phosphate-buffered saline to remove medium residues. Then spheroids were incubated in Krebs-Ringer buffer (107 mM NaCl (Sigma, USA), 5 mM KCl (Sigma, USA), 3 mM CaCl₂ (Sigma, USA), 1 mM MgSO₄*6H₂O (Sigma, USA), 7 mM NaHCO₃ (Sigma, USA), 0.1% BSA (Sigma, USA) and 20 mM HEPES (Sigma, USA), pH=7.6) at 37° C. for 2 hours. The buffer was removed and the spheroids were divided into 4 aliquots and incubated at 37° C. for 1 hour at different glucose concentrations (Sigma, USA) in Krebs-Ringer buffer: 0 mM, 2 mM, 5 mM and 15 mM glucose. Supernatant was collected in test tubes for further ELISA analysis of insulin and C-peptide content using the Ultrasensitive Insulin ELISA Kit (Mercodia, Sweden) and Ultrasensitive C-Peptide ELISA Kit (Mercodia, Sweden) according to the manufacturer's instructions. The result was analyzed using Start Fax-2100 plate reader (Awareness Technology, USA). Spheroids were analyzed for protein content by the Bradford Protein Assays method (Thermo Fisher Scientific, Germany) according to the manufacturer's instructions. The amount of protein was determined by Start Fax-2100 plate reader (Awareness Technology, USA) at a wavelength of 595 nm in a cuvette with a layer thickness of 10 mm. After that, the amount of secreted insulin and C-peptide was normalized by the total amount of protein. It was shown that human salivary gland cells acquired the ability for glucose-dependent insulin secretion after pancreatic differentiation in three-dimensional cultivation conditions. Insulin secretion increased at least twice (Table 6) with an increase in glucose concentration in the medium from 5 mM to 15 mM and reaches 14.5 ng/mg protein. Simultaneous secretion of C-peptide by cells in an equimolar amount with insulin confirmed the endogenous origin of the detected insulin and its proper maturation in the salivary gland cells after pancreatic differentiation (Table 6). Thus, human salivary gland cells possess the ability for glucose-dependent secretion in vitro in three-dimensional cultivation conditions after pancreatic differentiation.

TABLE 6
The dependence of insulin and C-peptide secretion by pancreatic cells
after pancreatic differentiation and three-dimensional cultivation on the
concentration of glucose in the medium. Concentrations of insulin and
C-peptide are given in ng/mg of protein per 1 hour of incubation of
the medium with the cells

| | Insulin | | C-peptide | |
|---|---|---|---|---|
| | Spheroids, | Spheroids, | Spheroids, | Spheroids, |
| Glucose | undifferentiated | cell | undifferentiated | cell |
| concentration | SGC | product | SGC | product |
| 0 mM | 0.49 | 6.01 | 0.38 | 2.16 |
| 2 mM | 0.46 | 9.24 | 0.33 | 2.96 |
| 5 mM | 0.5 | 8.26 | 0.26 | 2.97 |
| 15 mM | 0.59 | 14.49 | 0.55 | 8.03 |

### Example 4

Analysis of the Functional Activity of Cell Product

Salivary gland cells were differentiated during the first passage as described in Example 1, then washed twice with Versene solution, removed from the culture flask surface by trypsin as described in Example 1, pelleted at 200 g for 5 minutes, washed thrice with phosphate-buffered saline, after that the cells were counted by use of automated cell counter (Bio-Rad, USA). Subsequently, the phosphate-buffered saline was added to the cells in amount of 500 μl of buffer per 5 million cells. Aliquots of the final suspension containing 5 million cells of the cell product were injected intraperitoneally with sterile syringes to immunodeficient Nude mice. Nude mice were used as control and were injected with 500 μl of cell-free phosphate-buffered saline. Three days after the injection, the few of the mice pancreas was examined: the animals were anesthetized with isoflurane (Sigma-Aldrich, USA), their stomach was treated with alcohol and the skin and peritoneum were incised. The pancreas was extracted with forceps and subjected to cryopreservation in a medium for the preparation of OCT cryosections (CrioMount Histolab, Sweden). The pancreas cryosections were prepared with the use of Leica CM1900 cryostat (Leica, Switzerland) on Super Frost Plus slides (Menzel, Germany) with a section thickness of 10 μm.

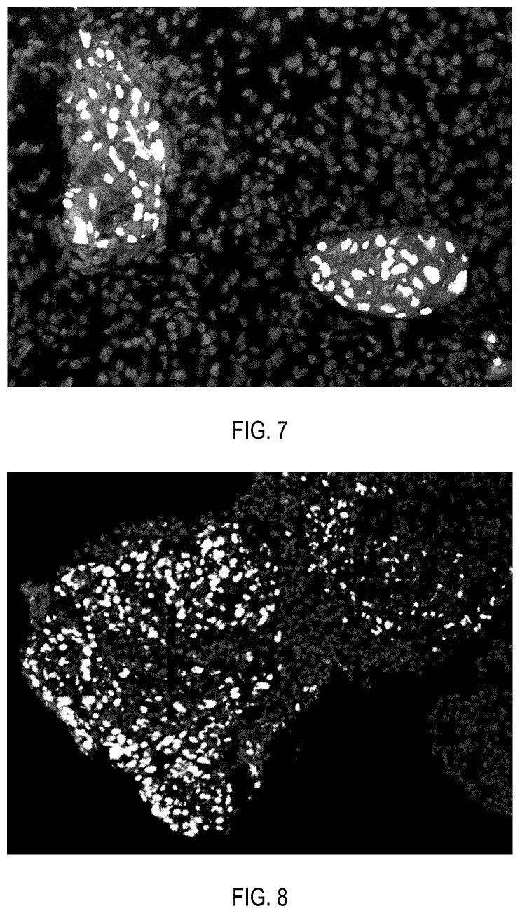

Immediately after preparation cryosections were fixed with 4% paraformaldehyde (Sigma, USA) at room temperature for 10 minutes. Then they were washed with phosphate-buffered saline and incubated with primary anti-Human nuclei antibodies conjugated to Alexa Fluor 488 fluorochrome and anti-insulin at a dilution of 1:500 in 0.3% triton solution (Sigma, C [symbol] A) and 2% fetal calf serum (Sigma, USA) in phosphate-buffered saline overnight at +4° C. Cryosections were then washed with phosphate-buffered saline three times for 10 minutes at a time at 37° C. and incubated with Alexa Fluor® 546 goat anti-rabbit IgG (H+L) (Invitrogen, USA) secondary antibodies at a dilution of 1:1000 in phosphate-buffered saline at 37° C. for 60 minutes. Cryosections were washed with phosphate-buffered saline three times for 10 minutes at a time and the cell nuclei were stained with 1 μg/ml DAPI (Sigma, USA) for 5 minutes. The cryosections were then washed with phosphate-buffered saline and enclosed under a cover glass in 25% glycerin (Sigma, USA) and 25% polyethylene glycol (Sigma, USA) medium in phosphate-buffered saline. The sections were analyzed under Keyence BZ-9000 fluorescent microscope (Keyence, Japan). In total, 5 experimental mice in each group of animals were analyzed. Transplanted cells were found in their pancreas where they formed aggregates (FIG. 7). Staining with antibodies to insulin confirmed these cells could produce insulin protein in vivo.

On the 7th day after cell transplantation, the content of human insulin in the blood serum of the remaining mice was analyzed. For this purpose, control mice that received 500 μl of phosphate-buffered saline and the experimental mice that received 5 million differentiated human salivary gland cells were subjected to starvation for 6 hours. After that, blood samples were taken from 5 control mice and 5 experimental mice: after anesthesia with isoflurane, the mice were exposed to the thorax opening and blood samples (0.5-1 ml) were taken from heart with a heparin-soaked syringe. The blood was placed in test tubes with heparin and pelleted at 10,000 rpm for 20 minutes. Blood serum was collected in separate tubes and frozen at −80° C. for further analysis. In addition, after a 6-hour starving, 5 control and 5 experimental mice were injected intraperitoneally with a solution of glucose in an amount of 2 mg per kg body mass. 30 minutes after the injection of glucose, blood samples were also taken and serum was prepared as described above.

An enzyme-linked immunosorbent assay for human insulin in the blood serum of mice was performed using the Ultrasensitive Insulin ELISA Kit (Mercodia, Sweden) according to the manufacturer's instructions. Start Fax-2100 plate reader (Awareness Technology, USA) was used for analysis. Human insulin was detected in the blood serum of all experimental mice from the test group. It should be noted that the amount of insulin in mice that were injected with glucose before taking blood for analysis was approximately two times greater (Table 7).

TABLE 7
The content of human insulin in the serum of experimental Nude mice.

| | Concentration of human insulin in |
|---|---|
| Animal groups | mice blood serum, pM |
| Control group | 0 |
| Experimental group | 0.6 |
| Experimental group after glucose | 1.3 |
| injection | |

The obtained result shows that after pancreatic differentiation human epithelial progenitor cells are capable of glucose-dependent insulin secretion in vivo after injection into the body.

It is known that salivary gland progenitor duct cells have the potential for differentiation into exocrine cells secreting amylase. Therefore, the cell ability for exocrine differentiation after pancreatic differentiation was studied. Human salivary gland cells at the first passage were subjected to endocrine pancreatic differentiation as described in Example 1. The cells were then immunocytochemically stained with antibodies to the salivary and pancreatic amylases by the method described above in Example 1. In addition, cryosections of spheroids were prepared and stained with antibodies to amylase using the methods described above in Example 3. Pancreas cryosections of immunodeficient Nude mice were stained with antibodies to amylase and Human nuclei on the third day after transplantation of 5 million human salivary gland cells (the method described above in Example 3).

Immunophenotypic analysis showed that amylase was not detected in human salivary gland cells either by cultivation on plastics or under three-dimensional culture conditions. In addition, human salivary gland cells did not acquire the ability to produce amylase in vivo—after transplantation to immunodeficient Nude mice. These results indicate the specificity of the endocrine differentiation of epithelial cells.

### Example 5

Analysis of Biological Safety of Human Salivary Gland Cells

Increased concentration of amylase in the body poses a hazard to health. Secretion potency of the human cells subjected to pancreatic differentiation was analyzed. For this purpose, salivary gland cell cultures were obtained from four 37-55 years old male donors. The cultures were named SGC-37M, SGC-50M, SGC-51 M and SGC-55M. In the first passage human salivary gland cells of all four cultures were seeded into 12-slot culture plates in amount of 1 million cells per slot. In 48 hours the culture medium was removed, the slots were washed thrice with phosphate-buffered saline, and then 1 ml of phosphate-buffered saline was added to the cells, providing further incubation at 37° C. for 2 hours. Buffer samples were collected and frozen at −80° C. for a subsequent ELISA. In addition, cells from all cultures were subjected to endocrine pancreatic differentiation as described above in Example 1, and spheroids were prepared by the method described in Example 3. In a similar way, buffer samples were collected from differentiated cells of the salivary gland and from spheroids.

All samples were analyzed by ELISA method for the salivary amylase content using Salivary Amylase Alpha 1 Human ELISA Kit (MyBioSource, USA) and the pancreatic amylase content using Pancreatic Amylase Human ELISA Kit according to the manufacturer's instructions. The result was analyzed using Start Fax-2100 plate reader (Awareness Technology, USA). As a positive control of the reaction, human blood serum (1—first donor, 2—second donor) was used.

ELISA method did not reveal the secretion of pancreatic amylase and salivary amylase in vitro by salivary gland cells of all four cultures. Amylase secretion was detected neither after pancreatic cell differentiation, nor under the three-dimensional culture conditions (Table 8). Amylase was not detected also in salivary gland cell lysate.

TABLE 8
ELISA of amylase for human salivary gland cells (SGC, control),
differentiated salivary gland cells (SGC-diff.) and differentiated
salivary gland cells in three-dimensional culture conditions
(SGC-spheroids), the first passage:

| | Salivary amylase, ng/ml | | | Pancreatic amylase, ng/ml | | |
|---|---|---|---|---|---|---|
| | | | SGC - | | | SGC - |
| | SGC, | SGC - | spher- | SGC, | SGC - | spher- |
| Cell culture | control | diff. | oids | control | diff. | oids |
| SGC-37M | 0 | 0 | 0 | 0 | 0 | 0 |
| SGC-50M | 0 | 0 | 0 | 0 | 0 | 0 |
| SGC-51M | 0 | 0 | 0 | 0 | 0 | 0 |
| KC [symbol] -55M | 0 | 0 | 0 | 0 | 0 | 0 |

| | | |
|---|---|---|
| Positive | 150 ng/ml | 100 ng/ml |
| Control 1 | | |
| Positive | 157.5 ng/ml | 105 ng/ml |
| Control 2 | | |

* human blood serum was used as a positive control, “1” - the first donor, “2” - the second donor.

For in vivo analysis of the biological safety of human epithelial cells, 5 million suspended differentiated salivary gland cells in 500 μl of phosphate-buffered saline were injected intraperitoneally to immunodeficient Nude mice. The mice injected with 500 μl of phosphate-buffered saline were used as controls. Seven days after transplantation, blood samples were taken from the mice for human serum amylase content analysis. Methods for obtaining cells, transplanting them, and obtaining blood serum from mice are described above in Examples 1 and 4. For ELISA, Salivary Amylase Alpha 1 Human ELISA Kit (MyBioSource, USA) and Pancreatic Amylase Human ELISA Kit (AbCam, UK) were used, the assay was performed according to manufacturers' instructions. The result was analyzed using Start Fax-2100 plate reader (Awareness Technology, USA). As a positive control of the reaction, human blood serum (1—first donor, 2—second donor) was used.

Enzyme-linked immunosorbent assay (ELISA) of Nude mice blood serum revealed no human amylase in the blood serum of mice 7 days after transplantation of differentiated human salivary gland cells, while amylase was detected in the positive control in amount of 100-150 ng/ml. This suggests that human salivary gland cells in vivo do not differentiate in the exocrine direction and do not produce amylase.

On the 3rd and 7th days after transplantation of human salivary gland cells into immunodeficient Nude mice, the histological analysis of the mice pancreas was carried out. For this purpose, the mice were anesthetized with isoflurane (Sigma-Aldrich, USA), their stomach was treated with alcohol and the skin and peritoneum were incised. The pancreas was extracted with forceps and fixed with 4% paraformaldehyde (Sigma, USA) at +4° C. for 60 minutes and used for preparation of histological specimen: the tissue was dehydrated with alcohols (70% ethanol (Sigma, USA) for 1 hour, 96% ethanol for 1 hour, 100% ethanol for 1 hour, 100% ethanol and chloroform (Sigma, USA) 1:1 for 10-15 minutes, chloroform for 10-15 minutes). Then the tissue was immersed in Histomix paraffin (Biovitrum, Russia), soaked for 1-2 hours ana prepared histological sections of the paraffin blocks were prepared using MICROM microtome (Carl Zeiss, Germany) with a section thickness of 5 μm. The preparations were then deparaffinized: xylene (Sigma, USA)—2 minutes, 100% ethanol (Sigma, USA)—2 minutes, 96% ethanol—2 minutes, 70% ethanol—2 minutes, the preparations were rinsed with distilled water. The sections were stained with hematoxylin (Sigma, USA) for 5 minutes, then rinsed with water and stained with eosin (Sigma, USA) for 40 seconds. The preparations were rinsed thrice with water and enclosed under a cover glass in 25% glycerin (Sigma, USA) and 25% polyethylene glycol (Sigma, USA) medium in phosphate-buffered saline. Sections were analyzed in transmitted light under Keyence BZ-9000 microscope (Keyence, Japan). Histological analysis of the pancreas of Nude mice with transplanted human cells revealed no pathological changes (fibrotic changes or inflammatory responses) in the pancreas of the studied mice on the 3^{rd} and 7^{th} days after transplantation compared to control mice injected with phosphate-buffered saline.

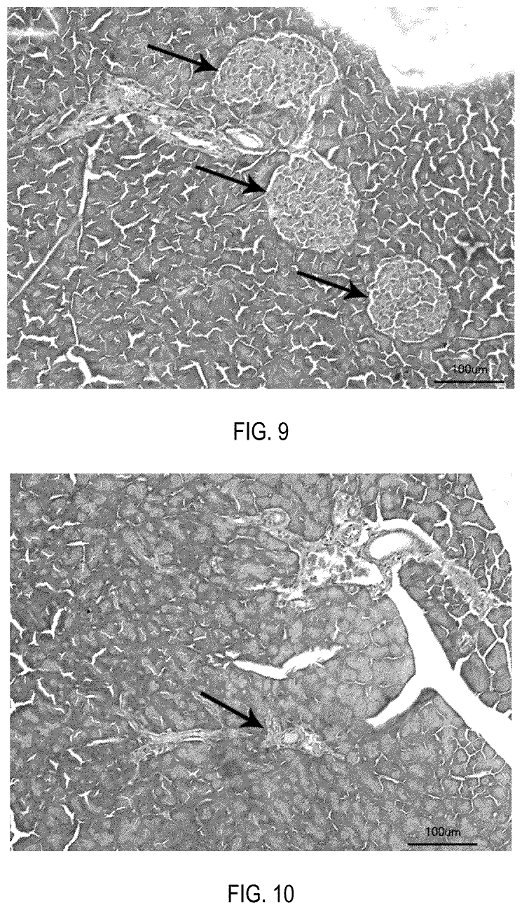

Pancreas of Nude mice (10 experimental and 10 control mice) were examined for CD68 macrophage marker on the third day after human cell transplantation in order to detect an inflammatory response. Cryosections were prepared and stained with antibodies to Human nuclei and CD68 as described in Example 4. Immunohistochemical staining showed no significant infiltration of macrophages due to transplantation of differentiated human salivary gland cells (FIG. 8)

To study the ability of human salivary gland cells to form tumors upon transplantation into the body after pancreatic differentiation, 10 million suspended differentiated human salivary gland cells in 500 μl of phosphate-buffered saline were transplanted subcutaneously into ten immunodeficient Nude mice during the first passage as described in Example 4. Ten Nude mice were used as a control and subcutaneously injected with 500 μl of cell-free phosphate-buffered saline. Over the next three months the mice survival rate and presence of tumors were observed. It was shown that after subcutaneous transplantation of 10 million differentiated human salivary gland cells into immunodeficient Nude mice, no death and signs of tumor formation were identified during all three months of observation.

Thus, it can be concluded that the tested human salivary gland cells conform to biological safety requirements: they do not form tumors, do not cause inflammatory reactions and pathological changes in tissues and do not produce potentially hazardous to health biologically active substances.

### Example 6

Correction of Experimental Diabetes in Mice

Experimental diabetes was induced in C57Black male mice (age: 6-8 weeks, weight: 20-22 g). After 8 hours of starvation, the animals were injected daily (for 5 consecutive days) intraperitoneally with streptozotocin (Sigma, USA) in amount of 40 μg per kg of body weight diluted in 200 μl of citrate buffer (Sigma, USA) ex tempore. The control group of animals was injected with the same amount of citrate buffer (Sigma, USA) (200 μl). The ordinary food and 10% glucose solution (Sigma, USA) in water were administered in the diet of animals for 5 days. Over the next week, the blood glucose level of animals was measured after 6 hours of starvation with OneTouch Select (Johnson & Johnson, USA) glucometer using OneTouch Ultra test strips (Johnson & Johnson, USA). To measure glucose in the blood of mice, a small incision at the tip of the tail was made and a drop of blood was placed on a test strip, the measurement was carried out with glucometer according to the manufacturer's instructions. 1 week after the last injection of streptozotocin, the animals had stable hyperglycemia with a blood glucose concentration of more than 25 mM. When stable hyperglycemia was established (7 days after the last injection of streptozotocin), the mice were divided into experimental and control groups. That day was considered a zero day of the experiment. On zero day of the experiment, 5 million of mouse salivary gland cells in 500 μl of phosphate-buffered saline, differentiated according to protocol described in Example 1, were transplanted into the animals of the test group, as described in Example 4. Diabetic animals injected with 500 μl of cell-free phosphate-buffered saline, as well as healthy non-diabetic animals also injected with 500 μl of cell-free phosphate-buffered saline were used as controls.

In the following 4 weeks of the experiment, the dynamics of glucose in the blood of mice was studied: the blood glucose level was measured in animals of all groups (at the same time after 4 hours of starvation, by the method described above). The first measurement was carried out on zero day of the experiment, then every 3 days. In addition, the survival of mice in all three experimental groups was assessed every day during two months.

Survival rate analysis showed that healthy mice (non-diabetic) did not die during two months, whereas the death in diabetic group was observed. As a result, about 14% of diabetic mice survived after two months of observation. At the same time, about 69% of the animals survived in the group of diabetic mice with transplanted salivary gland cells (Table 9). Thus, transplantation of differentiated salivary gland cells significantly increases the survival of mice with experimental diabetes.

TABLE 9
The survival rate of experimental mice, the proportion of mice
surviving is shown in percent, the number of animals in each
group at the beginning of the experiment - 17 pieces.

| | Experiment time, weeks | | | | | | |
|---|---|---|---|---|---|---|---|
| Animal groups | 0 | 1.4 | 1.86 | 2.14 | 2.86 | 7 | 9.14 |
| Healthy mice | 100 | 100 | 100 | 100 | 100 | 100 | 100 |
| Mice with | 100 | 100 | 85.7 | 57.14 | 28.57 | 14.29 | 14.29 |
| streptozotocin | | | | | | | |
| induced diabetes | | | | | | | |
| Mice with | 100 | 87.5 | 87.5 | 75 | 68.75 | 68.75 | 68.75 |
| streptozotocin | | | | | | | |
| induced diabetes, | | | | | | | |
| that received | | | | | | | |
| cell product | | | | | | | |
| transplantation | | | | | | | |

The blood glucose concentration of the experimental animals was evaluated for 30 days, as streptozotocin diabetes was stable during this time. It was shown that at the start of the experiment (day 0) in the group of control diabetic mice injected with 500 μl of cell-free phosphate-buffered saline and in the experimental group of mice with transplanted cells, the glucose concentration was about 27 mM, while in the group of healthy mice—about 6 mM. Cell transplantation had an ambivalent effect on the course of experimental diabetes in animals: firstly, after transplantation of differentiated salivary gland cells in mice, there are no sharp jumps of glucose that exist in the group of diabetic animals without cell transplantation (Table 10). Secondly, gradual decrease in blood glucose concentration is observed in animals after cell transplantation. At the end of the experiment (day 30) there was a statistically significant reduction in the blood glucose concentration to 11 mM in mice with transplanted cells, while the glucose concentration level remained high (about 22 mM) in diabetic mice without transplanted cells.

TABLE 10
The dynamics of the concentration of glucose in the blood of the
experimental mice, the concentration of glucose in the blood serum
is given in mm, the number of animals in each group is 10.

| | Days from the beginning of the experiment | | | | | | | | | |
|---|---|---|---|---|---|---|---|---|---|---|
| Animal groups | 0 | 5 | 7 | 9 | 12 | 16 | 21 | 23 | 28 | 30 |
| healty mice | 6 | 6.6 | 7.2 | 6.6 | 7.1 | 6.9 | 6.6 | 7 | 6 | 7.8 |
| Mice with | 27 | 24.1 | 23 | 23.3 | 14.2 | 16.3 | 19.6 | 23.7 | 20.2 | 22.1 |
| streptozotocin | | | | | | | | | | |
| induced diabetes | | | | | | | | | | |
| Mice with | 27 | 20.5 | 17.9 | 18 | 11.7 | 13.5 | 13.1 | 12.8 | 10.9 | 10.9 |
| streptozotocin | | | | | | | | | | |
| induced diabetes, | | | | | | | | | | |
| that received cell | | | | | | | | | | |
| product | | | | | | | | | | |
| transplantation | | | | | | | | | | |

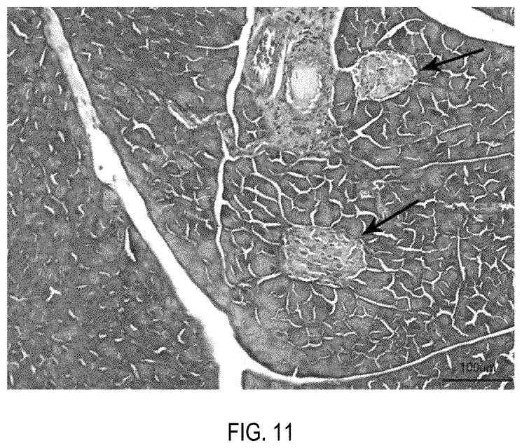

On the 40th day of the experiment, histological analysis of the mice pancreas in all three groups was carried out. Histological preparations from the pancreas were prepared as described above in Example 5. Analysis of mouse salivary gland serial sections showed that degradation of islets has occurred in diabetic mice which did not receive the cell transplantation (FIG. 10, indicated by arrows). In addition, there was an increase in vessel diameter of these animals. In diabetic mice received transplantation of 5 million differentiated salivary gland cells, regenerating islets were observed on the 40th day after transplantation (FIG. 11, indicated by arrows). At the same time, normal Langerhans islets are observed in normal animals without diabetes (FIG. 9, indicated by arrows). Thus, transplantation of salivary gland cells increases the survival rate of mice with experimental diabetes, reduces hyperglycemia and reduces jumps in blood glucose concentration in diabetic mice, and also promotes regeneration of the islets of Langerhans. In general, it can be concluded that the differentiated salivary gland cells contribute significantly to the correction of experimental diabetes evidenced from streptozotocin-induced diabetic model in mice.

### Example 7

Cultivation of Epithelial Progenitor Cells and Obtaining Cell Product of Insulin-Producing Cells

Bioptic sample of human submandibular salivary gland with a volume of 2 cm³ was obtained during a planned surgery to remove part of the salivary gland due to sialolithiasis. The donor of the material (male, 37 years old) was free from infections: hepatitis B and C viruses, HIV, syphilis.

Bioptic sample was immersed in sterile DMEM/F12 1:1 medium (Gibco, USA) with 40 g/ml gentamicin (Sigma, USA) and transported to the laboratory in a sealed container. Further manipulations were carried out in sterile conditions compliant with GMP requirements (Good Manufacturing Practice). Bioptic sample was immersed in Petri dish containing phosphate-buffered saline, the tissue was mechanically divided into 5 mm³ fragments by sterile scalpel and forceps, and adipose tissue was mechanically separated and removed. Then epithelial tissue fragments were washed twice with phosphate-buffered saline, pelleted at 200 g for 5 minutes and incubated with 2 mg/ml IV type collagenase (Gibco, USA) in DMEM/F12 1:1 medium (Gibco, USA) containing 2 mM glutamine (Invitrogen, USA) at 37° C. for 40 minutes. After that, the cell suspension was pipetted for 5 minutes, passed through a nylon filter with a pore diameter of 100 μm, and the cells were pelleted at 200 g for 10 minutes. The cells were washed twice with phosphate-buffered saline, pelleted at 200 g for 5 minutes. The cells were magnetically separated by EpCAM marker (Miltenyi Biotec GmbH, Germany) according to the manufacturer's instructions. For this purpose, the cells were counted by use of automated cell counter (Bio-Rad, USA) and resuspended in phosphate-buffered saline at a concentration of 5×10⁶ cells per ml of buffer. The cells were incubated at 4° C. for 20 minutes with antibodies in amount of 5 μg antibodies per 10⁶ cells. After that, the cells were magnetically separated on columns according to the manufacturer's instructions. Sorted out cells were pelleted during 5-10 minutes at 200 g, washed twice with phosphate-buffered saline and resuspended in PCT Epidermal Keratinocyte Medium (1×, liquid), (CELLnTEC, Switzerland), # CnT-07, containing 1×1× insulin-transferrin-selenite supplement (Invitrogen, USA) and 10 ng/ml epidermal growth factor (Sigma, USA). The cells were seeded into I type collagen coated culture flasks in amount of 5×10³ cells per cm² and incubated at 37° C. and 5% CO₂. The medium was changed every day during the first 5 days and every 3 days in further cultivation. These cells are a zero passage. As a result, 10 million epithelial progenitor cells expressing EpCAM were obtained from 2 cm³ bioptic sample.

These cells also expressed epithelial cell markers: KRT18 and CD49f, which was confirmed by flow cytometry. For this purpose, 2 million cells were fixed with 1% paraformaldehyde (Sigma, USA) at room temperature for 5 minutes, the cells were washed twice with phosphate-buffered saline and divided into 4 aliquots of 0.5 million in 200 μl of phosphate-buffered saline containing 1% bovine serum albumin (Sigma, USA) and 0.1% triton (Sigma, USA). Then Anti-KRT18 primary antibodies (AbCam, UK) were added to the cells of the first aliquot at a dilution of 1:500. Anti-CD49f primary antibodies (Millipore, USA) were added to the cells of the second aliquot at a dilution of 1:10. The corresponding isotype controls were added to the cells of the third and fourth aliquots. Cells with antibodies were incubated at 37° C. for 60 minutes. Then the cells were washed thrice with phosphate-buffered saline (for 10 minutes at a time, at 37° C.), resuspended in 200 μl of phosphate-buffered saline and incubated with secondary antibodies at 37° C. in darkness for 40 minutes. The cells were washed thrice with phosphate-buffered saline (for 10 minutes at a time, at 37° C.), resuspended in 1 ml of phosphate-buffered saline and analyzed by Cell Lab Quanta™ SC MPL flow cytometer (Beckman Coulter, USA). The relevant isotype control was used and at least 10,000 cells were analyzed. As a result, it was shown that more than 99% of the obtained epithelial progenitor cells of human salivary gland expressed KRT18 and CD49f markers.

After 10 days of cultivation, the cells of zero passage reached monolayer in amounts of 30 million. The cells were removed from the culture flask surface by trypsin. The culture medium was removed, the cells were washed twice with Versene solution, then incubated at 37° C. for 5 minutes with addition of 0.05% trypsin in amount of 1 ml per 25 cm² of a culture flask surface area. Then the cells were washed from trypsin by phosphate-buffered saline, pelleted at 200 g for 5-10 minutes. 15 million cells were cryogenically frozen for long-term storage, while the remaining 15 million cells were further cultivated to increase the cell mass. For this purpose, the first passage was provided: the cells were diluted in growth medium at the ratio of 1:5 and transferred in new I type collagen coated culture flasks. The culture medium was changed every 3 days. In 7 days the cells of the first passage reached monolayer in amounts of 75 million. The cells were removed from the culture flask surface by trypsin as described above, diluted in growth medium at the ratio of 1:3 and transferred in new I type collagen coated culture flasks. The culture medium was changed every 3 days. In 7 days the cells of the second passage reached monolayer in amounts of 225 million. Thus, the scale-up process was completed, proceeding to pancreatic cell differentiation.

Pancreatic differentiation of salivary gland epithelial cells was carried out according to the developed protocol: from day 0 to day 7 cells were incubated in DMEM/F12 1:1 medium (Gibco, USA) containing 10% fetal calf serum (HyClone, USA), 1× insulin-transferrin-selenite supplement (Invitrogen, USA), 2 mM glutamine (Invitrogen, USA), 10 ng/ml epidermal growth factor (Sigma, USA) with addition of 2 μm retinoic acid (Sigma, USA), 1 μm isoproterenol (Sigma, USA), 10 ng/ml fibroblast growth factor 10 (Life Technologies, USA) and 10 ng/ml insulin-like growth factor 1 (R&D, USA). From day 7 to day 14 cells were incubated in DMEM/F12 1:1 medium (Gibco, USA) containing 10% fetal calf serum (HyClone, USA), 2 mM glutamine (Invitrogen, USA), 10 ng/ml epidermal growth factor (Sigma, USA) with addition of 100 nM retinoic acid (Sigma, USA), 10 mM nicotinamide (Sigma, USA), 10 ng/ml insulin-like growth factor 1 (R&D, USA), 20 ng/ml hepatocyte growth factor (Gibco, USA) and 0.1 μm dexamethasone (Sigma, USA).

On the 14^{th} day of differentiation, total RNA isolation from cells, complementary DNA synthesis and pancreatic cell differentiation genetic markers analyses were carried out using qRT-PCR method as described in Example 1. As a result, it was shown that the differentiated human salivary gland cells expressed beta-cell markers: NGN3, PDX1, MAFA, PAX4, NKX6.1, as well as preproinsulin.

The flow cytometry method (described above) showed that 99% of these differentiated human salivary gland cells expressed KRT18 and CD49f epithelial cell markers and 89% expressed insulin.

For the analysis of glucose-dependent insulin secretion, spheroids of differentiated human salivary gland cells were prepared as described in Example 3 and Krebs-Ringer buffer samples were collected: from 0 mM, 2 mM, 5 mM and 15 mM glucose and incubated with spheroids for 1 hour as described in Example 3. Then ELISA analysis of insulin and C-peptide content was performed using the Ultrasensitive Insulin ELISA Kit (Mercodia, Sweden) and Ultrasensitive C-Peptide ELISA Kit (Mercodia, Sweden) according to the manufacturer's instructions. It was evidenced that the secretion of insulin and C-peptide increased at least twice with an increase in glucose concentration in the buffer from 5 mM to 15 mM.

The results of insulin ELISA for the three-dimensionally cultivated cell product with glucose concentration in the medium of 0 mM, 5 mM and 15 mM (after 5 hr of glucose addition) were 200 μM, 320 μM, and 550 μM, respectively.

The results of C-peptide ELISA for the three-dimensionally cultivated cell product with glucose concentration in the medium of 0 mM, 5 mM and 15 mM per 1 million cells were 150 μM, 200 μM, and 580 μM, respectively.

Thus, 225 million cells of the cell product containing cells capable of glucose-dependent insulin secretion and expressing NGN3, PDX1, MAFA, PAX4, NKX6.1 markers and preproinsulin were obtained. The product contains 89% of cells expressing insulin, KRT18 and CD49f and 11% of cells expressing KRT18 and CD49f.

### Example 8

Correction of Experimental Diabetes in Pigs

To obtain the cells of the submandibular salivary gland (CSF), the animals of the Ossabaw minipig line at the age of 3 months were used. Under general anesthesia, the submandibular salivary gland was removed, placed in a sterile container with DMEM/F12 medium 1:1 (Gibco, USA) with 40 μg/ml gentamicin (Sigma, USA), the biopsy was transported to the laboratory in less than 4 hours. Following work was conducted under sterile conditions. Washed gland was placed in a 100 mm dish with 10 ml DMEM/F12 1:1 (Gibco, USA) and 40 μg/ml gentamicin (Sigma, USA), the capsule was carefully removed, as well as blood vessels and adipose tissue. The purified gland was transferred to another 100 mm dish with 10 ml of DMEM/F12 1:1 medium (Gibco, USA) and 40 μg/ml gentamicin (Sigma, USA), and two fine tweezers were used to cut the gland into as small pieces as possible no more than 2 mm³). The slurry was transferred to a 15 ml tube and washed twice with phosphate buffered saline, pelleted by centrifugation for 5 minutes at 200 g. The supernatant was removed, the pellet was resuspended in DMEM/F12 1:1 medium (Gibco, USA) containing 2 mM glutamine (Invitrogen, USA) and 2 mg/ml collagenase type IV (Gibco, USA) at a rate of 3 ml of solution per 1 cm³ of tissue. The cell suspension was incubated in a CO₂ incubator at 37° C. for 1 hour (the suspension was shaken every 10 minutes). The suspension was then actively pipetted for 5 minutes, passed through a nylon filter, and precipitated by centrifugation for 2 minutes at 200 g. The supernatant was removed, the cells were resuspended in full growth medium DMEM/F12 1:1 (Gibco, USA) containing 10% fetal bovine serum (HyClone, USA), 1× insulin-transferrin-selenite (Invitrogen, USA), 2 mM glutamine Invitrogen, USA), 10 ng/ml epidermal growth factor (Sigma, USA). Cells were placed on type I collagen-coated culture flasks at a rate of 5×10³ cells per cm², incubated at 37° C. and 5% CO₂. During the first 5 days, the medium was changed every day, during further cultivation—every 3 days. After obtaining the required number of cells, they were subjected to pancreatic differentiation according to a standard protocol: from day 0 to day 7 cells were incubated in DMEM/F12 1:1 medium (Gibco, USA) containing 10% fetal bovine serum (HyClone, USA), 1× insulin-transferrin-selenite (Invitrogen, USA), 2 mM glutamine (Invitrogen, USA), 10 ng/ml epidermal growth factor (Sigma, USA), with 2 μM retinoic acid (Sigma, USA), 1 μM isoproterolol (Sigma, USA), 10 ng/ml fibroblast growth factor 10 (Life Technologies, USA) and 10 ng/ml insulin-like growth factor 1 (R & D, USA). From day 7 to day 14 cells are incubated in DMEM/F12 1:1 medium (Gibco, USA) with 10% fetal bovine serum (HyClone, USA), 2 mM glutamine (Invitrogen, USA), 10 ng/ml epidermal growth factor (Sigma (USA), 10 mM nicotinamide (Sigma, USA), 10 ng/ml insulin-like growth factor 1 (R & D, USA), 20 ng/ml hepatocyte growth factor (Gibco, USA), with the addition of 100 nM retinoic acid (Sigma, USA)) and 0.1 μM dexamethasone (Sigma, USA).

Streptozotocin model was used to induce experimental diabetes. Male minipigs of the Ossabaw line at the age of 3 months after 24 hour fasting were given a single intravenous injection of streptozotocin (Sigma, USA) in citrated buffer at a dose of 125 mg per kg of animal body weight (total 20 animals). For control, minipigs were used, which were administered citrate buffer without streptozotocin (5 animals in all, the “control” group). 72 hours after the injection, and then weekly for 4 weeks, the fasting glucose level in the fasting minipigs (after an 8-hour fasting) was examined using OneTouch Select (Johnson & Johnson, USA) and OneTouch Ultra (Johnson & Johnson, USA) test strips. It was confirmed that animals injected with streptozotocin had a blood glucose level of at least 16.7 mM (300 mg/dl), which indicates the development of diabetes in them. The control animals that received the citrate buffer had a normal glucose level (5-8 mM). After 4 weeks, minipigs with diabetes were randomly divided into two groups of 10 animals. One group received cell transplantation (group “experiment”). After 12 hours of fasting, the animals were intraperitoneally injected into the pancreas projection with 20 million differentiated swine salivary gland cells in 5 ml of phosphate-buffered saline. The second group of diabetes mice were injected with 5 ml of phosphate-buffered saline (group “diabetes”). A study of blood glucose levels in all three groups of animals (norm, experiment, diabetes) was performed after an 8-hour fasting weekly for the next 8 weeks.

TABLE 11
The level of glucose in the blood of minipigs, mM. The time of the
experiment is the number of weeks after the transplantation of pig
piglets. Groups of animals: control - minipigs without diabetes
(5 pieces), experiment - minipigs with induced diabetes
(10 pieces), which received differentiated salivary gland cell
transplantation, diabetes - minipigs with diabetes without the
transplantation (10 pieces)

| Experiment time | Animal groups | | |
|---|---|---|---|
| (weeks) | control | experiment | diabetes |
| 1 | 5.0 ± 0.3 | 17.6 ± 2.1 | 18.4 ± 2.4 |
| 2 | 5.1 ± 0.4 | 16.5 ± 3.4 | 18.5 ± 2.8 |
| 3 | 5.5 ± 0.3 | 15.3 ± 3.9 | 17.7 ± 3.0 |
| 4 | 5.0 ± 0.2 | 14.7 ± 2.8 | 17.9 ± 2.7 |
| 5 | 5.1 ± 0.3 | 12.2 ± 3.6 | 18.8 ± 2.3 |
| 6 | 5.6 ± 0.1 | 11.4 ± 1.3 | 18.6 ± 3.3 |
| 7 | 5.1 ± 0.2 | 8.9 ± 0.8 | 19.4 ± 3.1 |
| 8 | 5.4 ± 0.2 | 7.2 ± 0.5 | 17.9 ± 2.2 |

It was shown that animals in the control group have a normal blood glucose level (about 5 mM) throughout the observation period (Table 11). Animals receiving cell transplantation demonstrated a statistically significant decrease in blood glucose levels from 3 weeks after cell transplantation. By the end of the experiment, the blood glucose level of the experimental animals is approaching the normal value (about 7 mM). Animals of the diabetes group have a high blood glucose level throughout the observation period (about 18 mM). Thus, differentiated swine salivary gland cells are able to correct experimental streptozotocin diabetes in minipigs.

1. A method for obtaining cell product of human insulin-producing cells, comprising obtaining epithelial progenitor cells from epithelial tissue biopsy and their consequent pancreatic differentiation into cells capable of glucose-dependent insulin secretion in which pancreatic differentiation is carried out in two stages:
(a) at the first stage, cells are cultured during 4-15 days in a culture medium supplemented with at least blood serum of mammals and glutamine in the presence of at least the following additives: epidermal growth factor, transferrin, sodium selenite, retinoic acid, isoproterenol;
(b) at the second stage, cells are cultured during 4-15 days in a culture medium supplemented with at least blood serum of mammals and glutamine in the presence of at least the following additives: epidermal growth factor, retinoic acid, nicotinamide, hepatocyte growth factor, dexamethasone;
where at both stages, cells are cultured at 37° C. in the presence of 5% CO₂.

2. The method of claim 1, wherein the culture medium of the first stage contains blood serum—2-20 volume %, glutamine—not less than 1-4 mM, epidermal growth factor—1-300 ng/ml, transferrin—not less than 0.1 mcg/ml, sodium selenite—0.1-20 ng/ml, retinoic acid—0.1-20 μM, isoproterenol—0.1-10 μM,

3. The method of claim 1, wherein the culture medium of the first ctage contains following concentrations of components: blood serum—2-20 volume %, glutamine—not less than 1 mM, epidermal growth factor—1-300 ng/ml, transferrin—not less than 0.1 mcg/ml, sodium selenite—0.1-20 ng/ml, retinoic acid—10 nM-20 μM, nicotinamide—1-100 mM, hepatocyte growth factor—1-300 ng/ml, dexamethasone—0.01-5 μM.

4. The method of claim 1, wherein the culture medium of the first stage contains additionally at least one of the following components: insulin-like growth factor 1, fibroblast growth factor 10, fibroblast growth factor 4, keratinocyte growth factor.

5. The method of claim 1, wherein the culture medium of the second stage additionally contains insulin-like growth factor 1 and/or betacellulin.

6. The method of claim 1, wherein the epithelial progenitor cells are isolated from biopsy material from salivary gland or intestines or stomach or liver or pancreas.

7. The method of claim 1, wherein cultivation is carried out in the additional presence of 5% O₂.

8. The method of claim 1, wherein before the pancreatic differentiation epithelial progenitor cells cultivation is carried out in order to increase their biomass.

9. The method of claim 1, wherein cell product is additionally cultivated in three dimensional conditions, obtaining spheroids.

10. A Cell product of insulin-producing cells of the mammal obtained according to claim 1, wherein containing at least 1 million cells or 10 thousand spheroids in 1 ml of isotonic solution.

11. A method for replacement therapy of diabetes mellitus involving transplantation of the cell product of claim 10, wherein containing 50-200 million cells into body of the recipient suffering from diabetes mellitus.

12. The method of claim 11, wherein dissimilar in that the cell product transplantiert in the form of spheroids.

13. The method of claim 11, wherein dissimilar in that the transplantation of the cell product injected intraperitoneally in the projection of the pancreas, or the spleen or into the portal vein, or in the greater omentum.

14. The method of claim 11, wherein dissimilar in that the cell product is administered 2-5 times with an interval of 1-6 months.
